(19) United States
(12) Patent Application Publication
(10) Pub. No.: US 2024/0307392 A1
(43) Pub. Date: Sep. 19, 2024

(54) ERK1/2 AND EGFR INHIBITORS COMBINATION THERAPY

(71) Applicant: Erasca, Inc., San Diego, CA (US)

(72) Inventors: Robert Field SHOEMAKER, San Diego, CA (US); Erin Denise LEW, San Diego, CA (US); Wei LIN, San Diego, CA (US); Leslie Harris BRAIL, San Diego, CA (US); Leenus MARTIN, San Diego, CA (US); Jingchuan ZHANG, San Diego, CA (US); Joanne OH, San Diego, CA (US)

(21) Appl. No.: 18/570,830

(22) PCT Filed: Jun. 23, 2022
(86) PCT No.: PCT/US2022/034660

§ 371 Date: Dec. 15, 2023

Related U.S. Application Data
(60) Provisional application No. 63/321,605, filed on Mar. 18, 2022.
(60) Provisional application No. 63/279,877, filed on Nov. 16, 2021.
(60) Provisional application No. 63/277,547, filed on Nov. 9, 2021.
(60) Provisional application No. 63/236,635, filed on Aug. 24, 2021.
(60) Provisional application No. 63/214,765, filed on Jun. 24, 2021.

(51) Int. Cl. A61K 31/506; A61K 31/497; A61K 31/519; A61K 39/395; A61P 35/00

(52) U.S. Cl. CPC A61K 31/506; A61K 31/497; A61K 31/519; A61K 39/3955; A61P 35/00

(57) ABSTRACT

The present disclosure relates generally to the use of an ERK1/2 inhibitor in combination with a EGFR inhibitor for treating cancer, specifically solid tumors. The present disclosure also relates to the use of an ERK 1/2 inhibitor in combination with a EGFR inhibitor and a BRAF inhibitor for treating cancer, specifically solid tumors.

### CROSS-REFERENCE

This application claims the benefit of U.S. Provisional Application Ser. No. 63/214,765 filed Jun. 24, 2021, U.S. Provisional Application Ser. No. 63/236,635 filed Aug. 24, 2021, U.S. Provisional Application Ser. No. 63/277,547 filed Nov. 9, 2021. U. S. Provisional Application Ser. No. 63/279,877 filed Nov. 16, 2022, U.S. Provisional Application Ser. No. 63/321,605 filed Mar. 18, 2022, which are hereby incorporated by reference in their entirety.

### BACKGROUND

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

### SUMMARY

ERK1/2 appear to be activated by MEK through phosphorylation of both a threonine and a tyrosine residue, namely at Tyr204/187 and Thr202/185. Once activated. ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus, it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK1/2, epidermal growth factor receptor (EGFR), a transmembrane protein that is a receptor for members of the epidermal growth factor family of extracellular protein ligands, also operates upstream of the RAS pathway.

EGFR is a receptor tyrosine kinase (RTK) which is a protein that is embedded in the cell membrane and relay growth signals from the outside environment to the cell's internal machinery. At rest, this protein resides on the cell membrane as inactive monomers. Growth factors secreted by nearby cells bind to specific RTKs, such as growth factor EGF binding to EGFR, and cause these RTKs to dimerize. Dimerized RTKs activate one another through transphosphorylation of their intracellular regions. Intracellular proteins, such as adapter proteins, bind to these phosphorylated regions and propagate the pro-growth signals within the cell via one or more signaling pathways. Cells express a variety of RTKs so that environmental cues can be relayed to specific cell populations in specific contexts. EGFR mediates pro-growth signaling in skin and in the ducts and outer surfaces of many organs. Overactive RTK signaling can result in uncontrolled cell growth and survival that transforms normal cells into cancer cells.

The opportunity to target signal transduction pathways from multiple angles and potentially ameliorate feedback loops upstream of Ras via ERK1/2 and EGFR provides opportunities for developing methods that employ combination therapies.

The present embodiments disclosed herein generally relate to compositions and methods related to combination therapies to treat cancer utilizing an ERK1/2 inhibitor in conjunction with an EGFR inhibitor while providing an unexpected degree synergy.

Disclosed herein is a method of treating cancer in a subject in need thereof, the method comprising: administering to the subject in need thereof a therapeutically effective amount of
(i) compound 1:

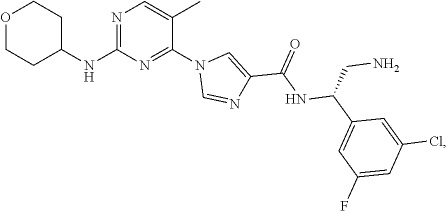 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) an EGFR inhibitor.

In some embodiments, the EGFR inhibitor is a small molecule EGFR inhibitor.

In some embodiments, the EGFR inhibitor is afatinib, amivantamab, canertinib, cetuximab, dacomitinib, daphnetin, erlotinib, gefitinib, icotinib, lapatinib, lazertinib, lifirafenib, mirzotamab clezutoclax, mobocertinib, nazartinib, necitumumab, neratinib, osimertinib, panitumamab, pelitinib, poziotinib, tivozanib, rociletinib, sapitinib, vandetanib, or varlitinib. In some embodiments, the EGFR inhibitor is osimertinib.

In some embodiments, osimertinib is administered in an amount that is about 80 mg/day.

In some embodiments, the EGFR inhibitor is cetuximab.

In some embodiments, cetuximab is initially administered at 400 mg/m² over 120 minutes followed by 250 mg/m² over 60) minutes once a week.

In some embodiments, cetuximab is administered at 500 mg/m² once every two weeks.

In some embodiments, cetuximab is administered at 400 mg/m² once every two weeks or 300 mg/m² once every two weeks.

In some embodiments, the method further comprises administering a BRAF inhibitor.

In some embodiments, the BRAF inhibitor is dabrafenib, encorafenib, regorafenib, sorafenib, or vemurafenib.

In some embodiments, the BRAF inhibitor is encorafenib.

In some embodiments, encorafenib is administered in an amount that is between about 100 mg/day and about 500 mg/day.

In some embodiments, encorafenib is administered in an amount that is about 450 mg/day.

In some embodiments, encorafenib is administered in an amount that is about 300 mg/day.

In some embodiments, encorafenib is administered in an amount that is about 225 mg/day

In some embodiments, encorafenib is administered in an amount that is about 150 mg/day. In some embodiments, the BRAF inhibitor is dabrafenib.

In some embodiments, dabrafenib is administered in an amount that is about 150 mg.

Also disclosed herein is a method of treating cancer in a subject in need thereof, the method comprising: administering to the subject in need thereof a therapeutically effective amount of
(i) compound 1:

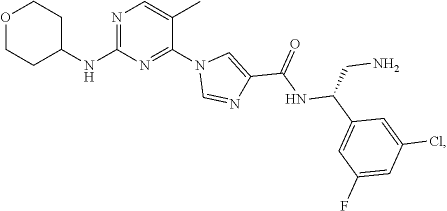 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) an EGFR inhibitor; and
(iii) a BRAF inhibitor

In some embodiments, the EGFR inhibitor is a small molecule inhibitor.

In some embodiments, the EGFR inhibitor is afatinib, amivantamab, cetuximab, dacomitinib, erlotinib, gefitinib, lapatinib, lazertinib, lifirafenib, mirzotamab clezutoclax, mobocertinib, nazartinib, necitumumab, neratinib, osimertinib, or vandetanib.

In some embodiments, the EGFR inhibitor is osimertinib

In some embodiments, osimertinib is administered in an amount that is about 80 mg/day.

In some embodiments, the EGFR inhibitor is cetuximab.

In some embodiments, cetuximab is initially administered at 400 mg/m² over 120 minutes followed by 250 mg/m² over 60 minutes once a week.

In some embodiments, cetuximab is administered at 500 mg/m² once every two weeks.

In some embodiments, cetuximab is administered at 400 mg/m² once every two weeks or 300 mg/m² once every two weeks.

In some embodiments, the BRAF inhibitor is dabrafenib, encorafenib, regorafenib, sorafenib, or vemurafenib.

In some embodiments, the BRAF inhibitor is encorafenib.

In some embodiments, encorafenib is administered in an amount that is between about 100 mg/day and about 500 mg/day.

In some embodiments, encorafenib is administered in an amount that is about 450 mg/day.

In some embodiments, encorafenib is administered in an amount that is about 300 mg/day. In some embodiments, encorafenib is administered in an amount that is about 225 mg/day.

In some embodiments, encorafenib is administered in an amount that is about 150 mg/day.

In some embodiments, the BRAF inhibitor is dabrafenib.

In some embodiments, dabrafenib is administered in an amount that is about 150 mg/day.

Also disclosed herein is a method of treating cancer in a subject in need thereof, the method comprising: administering to the subject in need thereof a therapeutically effective amount of
(i) compound 1:

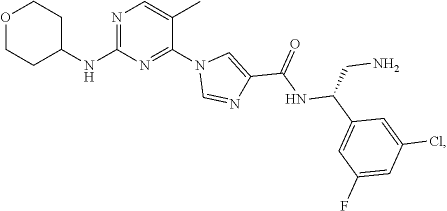 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) cetuximab; and
(iii) encorafenib.

A method of treating cancer in a subject in need thereof, the method comprising administering to the subject in need thereof a therapeutically effective amount of
(i) compound 1:

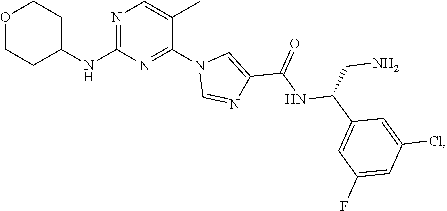 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) cetuximab; and
(iii) dabrafenib.

Also disclosed herein is a method of treating cancer in a subject in need thereof, the method comprising administering to the subject in need thereof a therapeutically effective amount of
(i) compound 1:

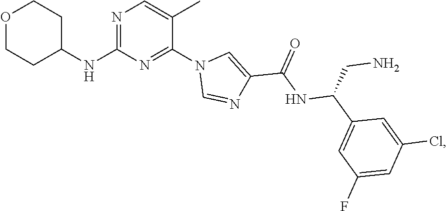 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) osimertinib; and
(iii) encorafenib.

Also disclosed herein is a method of treating cancer in a subject in need thereof, the method comprising administering to the subject in need thereof a therapeutically effective amount of
(i) compound 1:

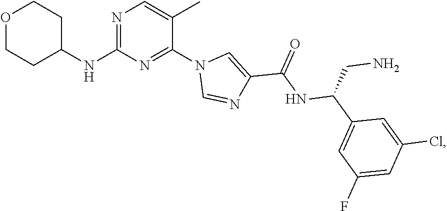 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) osimertinib: and
(iii) dabrafenib.

In some embodiments, the pharmaceutically acceptable salt of compound 1 is the mandelic acid salt.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation.

In some embodiments, the cancer has a G12V KRAS mutation. In some embodiments, the cancer has a G13D KRAS mutation. In some embodiments, the cancer has a Q16H KRAS mutation.

In some embodiments, the cancer has a Q16K KRAS mutation. In some embodiments, the cancer has a Q61R NRAS mutation.

In some embodiments, the cancer is a BRAF V600E or V600K mutant tumor.

In some embodiments, the cancer is a MAPKm/MAPKi-naïve pan cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

In some embodiments, the cancer comprises one or more EGFR mutation selected from the group consisting of L858R, exon 19 deletion, and T790M.

In some embodiments, the cancer is a solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the NSCLC is a NRAS Q61R mutant NSCLC.

In some embodiments, the cancer is a MAPKm/MAPKi-naïve NSCLC.

In some embodiments, the cancer is a BRAFi-treated V600 NSCLC.

In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC.

In some embodiments, the cancer is pancreatic cancer.

In some embodiments, the cancer is a MAPKm/MAPKi-naïve pancreatic cancer.

In some embodiments, the cancer is melanoma.

In some embodiments, the melanoma is a BRAF V600E or V600K mutant tumor.

In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer is esophageal cancer.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day. or about 250 mg/day.

In some embodiments, compound 1, or a pharmaceutically acceptable salt thereof, is administered in an amount that is about 25 mg/day, about 50 mg/day, about 100 mg/day, or about 150) mg/day.

In some embodiments, compound 1, or a pharmaceutically acceptable salt thereof, is administered in an amount that is about 250 mg/day.

In some embodiments, compound 1, or a pharmaceutically acceptable salt thereof, is administered once a day (QD). In some embodiments, compound 1, or a pharmaceutically acceptable salt thereof, is administered twice a day (BID). In some embodiments, compound 1, or a pharmaceutically acceptable salt thereof, is administered three times a day (TID).

In some embodiments, compound 1, or a pharmaceutically acceptable salt thereof, is administered once a week. In some embodiments, compound 1, or a pharmaceutically acceptable salt thereof, is administered twice a week.

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg. 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered for at least one 28-day cycle.

In some embodiments, compound 1, or a pharmaceutically acceptable salt thereof, is administered on day 1, day 8, day 15, and day 22 of a 28-day cycle.

In some embodiments, compound 1, or a pharmaceutically acceptable salt thereof, is administered on day 1, day 8, day 15 of a 28-day cycle.

In some embodiments, compound 1, or a pharmaceutically acceptable salt thereof, is administered orally.

In some embodiments, the method further comprises administering an additional MAPK pathway inhibitor. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. In some embodiments, the additional MAPK pathway inhibitor is a KRAS inhibitor. In some embodiments, the additional MAPK pathway inhibitor is a BRAF inhibitor. In some embodiments, the additional MAPK pathway inhibitor is a EGFR inhibitor. In some embodiments, the additional MAPK pathway inhibitor is a CDK4/6. In some embodiments, the additional MAPK pathway inhibitor is a FLT3 inhibitor. In some embodiments, the additional MAPK pathway inhibitor is adagrasib, afatinib. ASTX029, binimetinib, cobimetinib, dacomitinib, erlotinib, gefitinib, gilteritinib, lapatinib, LTT462. LY3214996, necitumumab, neratinib, nimotuzumab, palbociclib, selumetinib, sotorasib, trametinib. ulixertinib, vandetanib, or vemurafenib. In some embodiments, the additional MAPK pathway inhibitor is adagrasib. In some embodiments, the additional MAPK pathway inhibitor is gilteritinib. In some embodiments, the additional MAPK pathway inhibitor is palbociclib. In some embodiments, the additional MAPK pathway inhibitor is panitumumab. In some embodiments, the additional MAPK pathway inhibitor is sotorasib.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows in-vivo data for compound 1+encorafenib and compound 1+encorafenib/cetuximab in encorafenib/cetuximab refractory-BRAF^{V600E} RKO CDX models.

FIG. 1B shows in-vivo data for compound 1+encorafenib and compound 1+encorafenib/cetuximab in encorafenib/cetuximab refractory-BRAF^{V600E} WiDr CDX models.

FIG. 2A shows in-vivo data for compound 1+Osimertinib in osimertinib refractory EGFR mutant PDX model (LUN2355-214).

FIG. 2B shows in-vivo data for compound 1+Osimertinib in osimertinib refractory EGFR mutant PDX model (LUN2355-128-33).

FIG. 3A shows in-vivo tumor growth curves for compound 1 alone, encorafenib alone, and compound 1+encorafenib in encorafenib refractory-BRAF V600E CR0004 PDX models.

FIG. 3B shows in-vivo tumor growth curves for compound 1 alone, encorafenib alone. cetuximab alone, compound 1+encorafenib combination, compound 1+encorafenib+cetuximab triple combination, and encorafenib+cetuximab combination in encorafenib+cetuximab (EC) refractory—BRAF V600E CR0004 PDX models.

FIG. 4A shows in-vivo tumor growth curves for compound 1 alone, encorafenib alone, and compound 1+encorafenib combination in encorafenib refractory-BRAF V600E CRC1011 PDX models.

FIG. 4B shows in-vivo tumor growth curves for compound 1 alone, encorafenib alone. cetuximab alone, compound 1+encorafenib combination, compound 1+encorafenib+cetuximab triple combination, and encorafenib+cetuximab combination in encorafenib+cetuximab (EC) refractory—BRAF V600E CRC1011 PDX models.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition. method, or process, or the like, described herein. “consist of” or “consist essentially of” the described features.

As used in the specification and appended claims, unless specified to the contrary, the following terms have the meaning indicated below.

As used herein, the term “therapeutic” means an agent utilized to treat, combat, ameliorate. prevent, or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue: providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient.” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue. system, animal, individual or human that is being sought by a researcher, veterinarian, medical doctor or other clinician, which includes one or more of the following: (1) preventing the disease: for example. preventing a disease, condition or disorder in an individual that may be predisposed to the disease. condition or disorder but does not yet experience or display the pathology or symptomatology of the disease, (2) inhibiting the disease: for example, inhibiting a disease, condition or disorder in an individual that is experiencing or displaying the pathology or symptomatology of the disease, condition or disorder (i.e., arresting further development of the pathology and/or symptomatology), and (3) ameliorating the disease: for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments. wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease: stabilization (i.e., not worsening) of the state of the condition. disorder or disease: delay in onset or slowing of the progression of the condition, disorder or disease: amelioration of the condition, disorder or disease state: and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compound 1

Disclosed herein is (S)-N-(2-amino-1-(3-chloro-5-fluorophenyl)ethyl)-1(5-methyl-2-((tetrahydro-2H-pyran-4-yl)amino)pyrimidin-4-yl)-1H-imidazole-4- carboxamide:

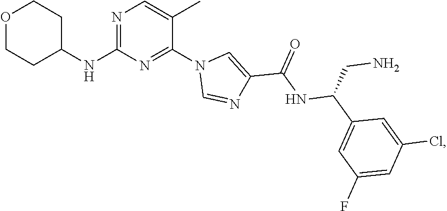 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound I is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

### EGFR Inhibitors

EGFR inhibitors are agents that bind to EGFR and slow down or stop cell growth, and can be classified as either tyrosine kinase inhibitors (TKI) or monoclonal antibodies.

TKIs are inhibitors that bind to the tyrosine kinase domain in the epidermal growth factor receptor and stop the activity of the EGFR. Examples include, without limitation, afatinib, dacomitinib, erlotinib, gefitinib, lapatinib, lazertinib, lifirafenib, mobocertinib, nazartinib, neratinib, osimertinib, and vandetanib.

Monoclonal antibody inhibitors are agents that bind to the extracellular component of the EGFR and prevent epidermal growth factor from binding to its own receptor, thus preventing cell division. Examples include, without limitation amivantamab, cetuximab, mirzotamab, clezutoclax, nimotuzumab, and necitumumab.

In some embodiments, the EGFR inhibitor is afatinib, amivantamab, canertinib, cetuximab, dacomitinib, daphnetin, erlotinib, gefitinib, icotinib, lapatinib, lazertinib, lifirafenib, mirzotamab clezutoclax, mobocertinib, nazartinib, necitumumab, neratinib, osimertinib, panitumamab, pelitinib, poziotinib, tivozanib, rociletinib, sapitinib, vandetanib, or varlitinib.

In some embodiments, the EGFR inhibitor is AC480, AEE788, AG-1478, AG-18, AG-490, AST-1306, AV-412, AZ5104, AZD3759, BIBX 1382, CGP-52411, CL-387785, CNX-2006, CUDC-101, OSI-420, PD153035 HCl, PD168393, TAK-285, Tyrphostin 9, Tyrphostin AG 183, WHI-P154, WHI-P180, WZ3146, or WZ4002.

In some embodiments, the EGFR inhibitor is a small molecule inhibitor.

In some embodiments, the EGFR inhibitor is osimertinib.

In some embodiments, the EGFR inhibitor is cetuximab.

In some embodiments, the EGFR inhibitor is afatinib.

In some embodiments, the EGFR inhibitor is dacomitinib.

In some embodiments, the EGFR inhibitor is erlotinib.

In some embodiments, the EGFR inhibitor is gefitinib.

In some embodiments, the EGFR inhibitor is lapatinib.

In some embodiments, the EGFR inhibitor is lazertinib.

In some embodiments, the EGFR inhibitor is lifirafenib.

In some embodiments, the EGFR inhibitor is mobocertinib.

In some embodiments, the EGFR inhibitor is nazartinib.

In some embodiments, the EGFR inhibitor is neratinib.

In some embodiments, the EGFR inhibitor is vandetanib.

In some embodiments, the EGFR inhibitor is not an anti-EGFR antibody inhibitor.

### Osimertinib

Osimertinib

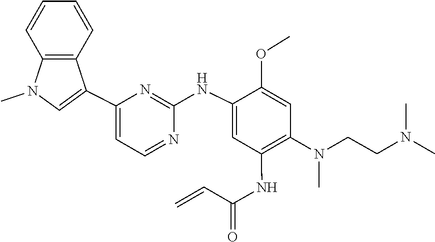

is a small molecule EGFR tyrosine kinase inhibitor and is used to treat locally advanced or metastatic NSCLC. It was approved by the FDA in November 2015 for the specific treatments of metastatic NSCLC with EGFR exon 19 deletions or exon 21 L858R mutations and EGFR T790M mutation-positive NSCLC. Osimertinib is sold as Tagrisso® by AstraZeneca.

### Cetuximab

Cetuximab is a chimeric monoclonal antibody EGFR inhibitor used for the treatment of metastatic CRC and head and neck cancer. In July 2009, cetuximab was approved by the FDA for treatment of colon cancer with wild-type KRAS. Cetuximab is sold as Erbitux® by Eli Lilly and Company.

### BRAF Inhibitors

BRAF inhibitors selectively target BRAF kinase and thus interfere with the MAPK signaling pathway that regulates the proliferation and survival of melanoma cells. BRAF inhibitors also have beneficial effects on the tumor microenvironment and anti-tumor immune response in BRAF-mutant melanoma, thus exerting immunomodulatory effects on the MAPK pathways and promote recognition of the tumor cells by the immune system and enhance anti-tumor T-cell response.

In some embodiments, the BRAF inhibitor is Encorafenib. In some embodiments, the BRAF inhibitor is Dabrafenib.

### Encorafenib

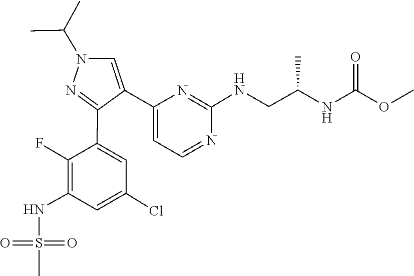 [SMILES: COC(=O)N[C@@H](C)CNc1nccc(-c2cn(C(C)C)nc2-c2cc(Cl)cc(NS(C)(=O)=O)c2F)n1]

Encorafenib is a drug for the treatment of certain

melanomas. It is a small molecule BRAF inhibitor that targets key enzymes in the MAPK signaling pathway. This pathway occurs in many different cancers including melanoma and colorectal cancers. In June 2018, it was approved by the FDA in combination with binimetinib for the treatment of patients with unresectable or metastatic BRAF V600E or V600K mutation-positive melanoma. Encorafenibis sold as Braftovi® by Pfizer.

### Dabrafenib

Dabrafenib

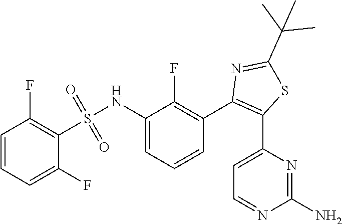 [SMILES: CC(C)(C)c1nc(-c2cccc(NS(=O)(=O)c3c(F)cccc3F)c2F)c(-c2ccnc(N)n2)s1]

is a medication for the treatment of cancers associated with a mutated version of the gene BRAF. Dabrafenib acts as an inhibitor of the associated enzyme B-Raf, which plays a role in the regulation of cell growth. Dabrafenib has clinical activity with a manageable safety profile in clinical trials of phase 1 and 2 in patients with BRAF (V600)-mutated metastatic melanoma. Dabrafenib is sold as Tafinlar® by Novartis.

### Combinations

Disclosed herein is a method of treating cancer in a subject in need thereof, the method comprising: administering to the subject in need thereof a therapeutically effective amount of
(i) compound 1:

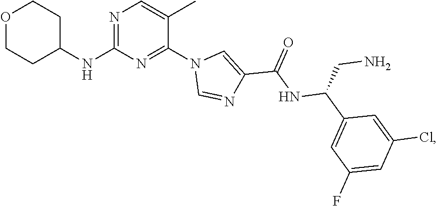 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) an EGFR inhibitor.

Disclosed herein is a method of treating cancer in a subject in need thereof, the method comprising: administering to the subject in need thereof a therapeutically effective amount of
(i) compound 1:

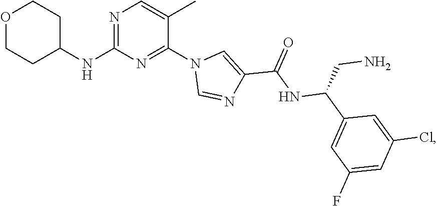 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) osimertinib.

Disclosed herein is a method of treating cancer in a subject in need thereof, the method comprising: administering to the subject in need thereof a therapeutically effective amount of
(i) compound 1:

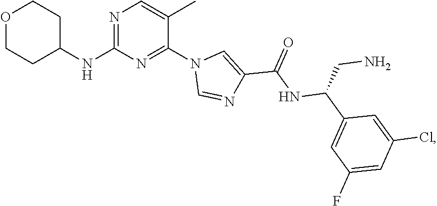 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) cetuximab.

Disclosed herein is a method of treating cancer in a subject in need thereof, the method comprising: administering to the subject in need thereof a therapeutically effective amount of
(i) compound 1:

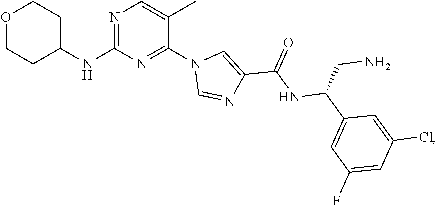 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) an EGFR inhibitor; and
(iii) a BRAF inhibitor.

In some embodiments, the EGFR inhibitor is a small molecule inhibitor.

In some embodiments, the EGFR inhibitor is afatinib, amivantamab, cetuximab, dacomitinib, erlotinib, gefitinib, lapatinib, lazertinib, lifirafenib, mirzotamab clezutoclax, mobocertinib, nazartinib, necitumumab, neratinib, osimertinib, nimotuzumab, or vandetanib. In some embodiments, the EGFR inhibitor is osimertinib. In some embodiments, the EGFR inhibitor is afatinib. In some embodiments, the EGFR inhibitor is dacomitinib. In some embodiments, the EGFR inhibitor is erlotinib. In some embodiments, the EGFR inhibitor is gefitinib. In some embodiments, the EGFR inhibitor is lapatinib. In some embodiments, the EGFR inhibitor is lazertinib. In some embodiments, the EGFR inhibitor is lifirafenib. In some embodiments, the EGFR inhibitor is mobocertinib. In some embodiments, the EGFR inhibitor is nazartinib. In some embodiments, the EGFR inhibitor is neratinib. In some embodiments, the EGFR inhibitor is vandetanib. In some embodiments, the EGFR inhibitor is cetuximab. In some embodiments, the EGFR inhibitor is not an anti-EGFR antibody inhibitor. In some embodiments, the BRAF inhibitor is dabrafenib, encorafenib, regorafenib, sorafenib, or vemurafenib.

In some embodiments, the BRAF inhibitor is dabrafenib. In some embodiments, the BRAF inhibitor is encorafenib.

Disclosed herein is a method of treating cancer in a subject in need thereof, the method comprising: administering to the subject in need thereof a therapeutically effective amount of
(i) compound 1:

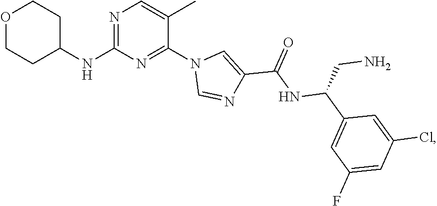 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) cetuximab; and
(iii) encorafenib.

Disclosed herein is a method of treating cancer in a subject in need thereof, the method comprising: administering to the subject in need thereof a therapeutically effective amount of
(i) compound 1:

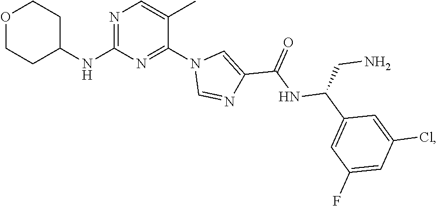 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) cetuximab: and
(iii) dabrafenib.

Disclosed herein is a method of treating cancer in a subject in need thereof, the method comprising: administering to the subject in need thereof a therapeutically effective amount of
(i) compound 1:

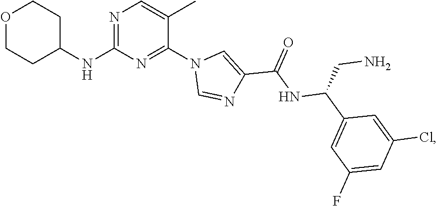 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) osimertinib; and
(iii) encorafenib.

Disclosed herein is a method of treating cancer in a subject in need thereof, the method comprising: administering to the subject in need thereof a therapeutically effective amount of
(i) compound 1:

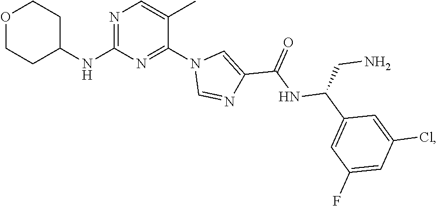 [SMILES: Cc1cnc(NC2CCOCC2)nc1-n1cnc(C(=O)N[C@H](CN)c2cc(F)cc(Cl)c2)c1]

or a pharmaceutically acceptable salt thereof,
(ii) osimertinib; and
(iii) dabrafenib.

### Further Combination

In some embodiments, the method comprises administering an additional MAPK pathway inhibitor. Without being bound by theory, suppression of MAPK signaling in cancer cells can result in downregulation of PD-LI expression and increase the likelihood that the cancer cells are detected by the immune system. Such third MAPK pathway inhibitors may be based on other mutations of proteins in the MAPK pathway. In some embodiments, the additional MAPK pathway inhibitor inhibits a protein in the MAPK pathway. In some embodiments, the additional MAPK pathway inhibitor inhibits a protein outside the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFRIR inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, ASTX029, binimetinib, cobimetinib, dacomitinib, erlotinib, gefitinib, gilteritinib, lapatinib, LTT462, LY3214996, necitumumab, neratinib, nimotuzumab, palbociclib, selumetinib, sotorasib, trametinib, ulixertinib, vandetanib, and vemurafenib.

In some embodiment the additional MAPK pathway inhibitor is adagrasib. In some embodiment the additional MAPK pathway inhibitor is afatinib. In some embodiment the additional MAPK pathway inhibitors is binimetinib. In some embodiment the additional MAPK pathway inhibitor is cobimetinib. In some embodiment the additional MAPK pathway inhibitor is dacomitinib. In some embodiment the additional MAPK pathway inhibitor is erlotinib. In some embodiment the additional MAPK pathway inhibitor is gefitinib. In some embodiment the additional MAPK pathway inhibitor is gilteritinib. In some embodiment the additional MAPK pathway inhibitor is lapatinib. In some embodiment the additional MAPK pathway inhibitor is LTT462. In some embodiment the additional MAPK pathway inhibitor is LY3214996. In some embodiment the additional MAPK pathway inhibitor is necitumumab. In some embodiment the additional MAPK pathway inhibitor is neratinib. In some embodiment the additional MAPK pathway inhibitor is nimotuzumab. In some embodiment the additional MAPK pathway inhibitor is palbociclib. In some embodiment the additional MAPK pathway inhibitor is selumetinib. In some embodiment the additional MAPK pathway inhibitor is sotorasib. In some embodiment the additional MAPK pathway inhibitor is trametinib. In some embodiment the additional MAPK pathway inhibitor is ulixertinib. In some embodiment the additional MAPK pathway inhibitor is vandetanib.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer. glioma, glioblastoma, neuroblastoma, prostate cancer, colorectal cancer, pancreatic cancer (such as pancreatic adenocarcinoma. PDAC), medulloblastoma, melanoma, cervical cancer, gastric cancer. ovarian cancer, lung cancer, cancer of the head. Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary. pancreas, rectum, stomach, and uterus. Additional examples include, thyroid carcinoma. cholangiocarcinoma, pancreatic adenocarcinoma, skin cutaneous melanoma, colon adenocarcinoma. rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme. ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer. malignant hypercalcemia, endometrial cancer, adrenal cortical cancer, neoplasms of the endocrine or exocrine pancreas, medullary thyroid cancer, medullary thyroid carcinoma, melanoma, colorectal cancer. papillary thyroid cancer, hepatocellular carcinoma, or prostate cancer.

In some embodiments, the cancer has a class 1 B-Raf mutation.

In some embodiments, the cancer harbors at least one of a EGFR. KRAS. BRAF (e.g., BRAF class III) and/or NF1 (e.g., loss of function) mutations.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer. HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61R NRAS mutation.

In some embodiments, the cancer is a MAPKm/MAPKi-naïve pan cancer.

In some embodiments, the cancer comprises one or more EGFR mutation selected from the group consisting of EGFR gene copy gain. EGFR gene amplification, chromosome 7 polysomy, L858R, exon 19 deletions/insertions. L718Q, L861Q, G719C, G719S, G719A, G724S, V765A, T783A, exon 20 insertions. EGFR splice variants (Viii, Vvi, and Vii), A289D, A289T, A289V, G598A, G598V, T790M, C797X, C797S, and S7681. In some embodiments, the cancer comprises one or more EGFR mutation selected from the group consisting of L858R, exon 19 deletion, and T790M.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer. gallbladder cancer, liver cancer, pancreatic cancer, small intestine cancer, or stomach cancer (gastric cancer).

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC.

In some embodiments, the NSCLC is a NRAS Q61R mutant NSCLC. In some embodiments. the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC.

In some embodiments, the cancer is pancreatic cancer. In some embodiments, the cancer is a MAPKm/MAPKi-naïve pancreatic cancer. In some embodiments, the cancer is pancreatic ductal adenocarcinoma (PDAC). In some embodiments, the PDAC is indicated by a KRAS G12V mutation.

In some embodiments, the cancer is melanoma. In some embodiments, the melanoma is a BRAF V600E or V600K mutant tumor. In some embodiments, the cancer is a BRAFi-treated V600) melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer is esophageal cancer.

In some embodiments, the cancer has one or more acquired mutations. In some embodiments. the acquired mutation results from a first-line treatment. In some embodiments, the first-line treatment is an EGFR inhibitor. In some embodiments, the EGFR inhibitor is osimertinib. In some embodiments, the EGFR inhibitor is cetuximab. In some embodiments, the cancer is a solid tumor cancer. In some embodiments, the cancer is NSCLC.

In some embodiments, the acquired mutation is an acquired EGFR mutation. In some embodiments, the acquired EGFR mutation is C797X. In some embodiments, the acquired EGFR mutation is L718Q. In some embodiments, the acquired EGFR mutation is EGFR amplification. In some embodiments, the acquired EGFR mutation is G724S. In some embodiments, the acquired mutation is S768I.

In some embodiments, the acquired mutation is an acquired amplification mutation. In some embodiments, the acquired mutation is a MET gene amplification. In some embodiments, the acquired mutation is HER2 gene amplification.

In some embodiments, the acquired mutation is an acquired oncogenic fusion. In some embodiments, the acquired oncogenic fusion is SPTBNI-ALK. In some embodiments, the acquired oncogenic fusion is RET fusion. In some embodiments, the acquired oncogenic fusion is BRAF fusion.

In some embodiments, the acquired mutation is an acquired MAPK-PI3K mutation. In some embodiments, the acquired MAPK-PI3K mutation is BRAF-V600E. In some embodiments, the acquired MAPK-PI3K mutation is PI3KCA. In some embodiments, the acquired MAPK-PI3K mutation is KRAS. In some embodiments, the acquired MAPK-PI3K mutation is HER2.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50) (the dose lethal to 50% of the population) and the ED50) (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, encorafenib is administered in an amount that is between about 100 mg/day and 500 mg/day. In some embodiments, encorafenib is administered in an amount that is about 450 mg/day. In some embodiments, encorafenib is administered in an amount that is about 300 mg/day. In some embodiments, encorafenib is administered in an amount that is about 250 mg/day. In some embodiments, encorafenib is administered in an amount that is about 225 mg/day. In some embodiments. encorafenib is administered in an amount that is about 200 mg/day. In some embodiments, encorafenib is administered in an amount that is about 150 mg/day.

In some embodiments, cetuximab is administered at 400 mg/m2 over 120 minutes followed by 250) mg/m2 over 60) minutes once a week. In some embodiments, cetuximab is administered at 500 mg/m2 once every two weeks. In some embodiments, cetuximab is administered at 400 mg/m2 once every two weeks. In some embodiments, cetuximab is administered at 300 mg/m2 once every two weeks. In some embodiments, cetuximab is administered at 200 mg/m2 once every two weeks.

In some embodiments, the amount of compound 1, or a pharmaceutically acceptable salt thereof, as described herein is relative to the free-base equivalent of compound 1.

In some embodiments, compound 1, or a pharmaceutically acceptable salt thereof, is administered orally.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day, about 150 mg/day., about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 mg twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is

administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW). In some embodiments, compound 1, or a pharmaceutically acceptable salt thereof, is administered in an amount that is between about 25 mg and about 300 mg once a week (QW).

In some embodiments, compound 1, or a pharmaceutically acceptable salt thereof, is administered in an amount that is between about 50 mg and about 250 mg once a week (QW).

In some embodiments, compound 1, or a pharmaceutically acceptable salt thereof, is administered in an amount that is between about 100 mg and about 300 mg once a week (QW).

In some embodiments, compound 1, or a pharmaceutically acceptable salt thereof, is administered in an amount that is between about 100 mg and about 250 mg once a week (QW).

In some embodiments, compound 1, or a pharmaceutically acceptable salt thereof, is administered in an amount that is between about 150 mg and about 300 mg once a week (QW).

In some embodiments, compound 1, or a pharmaceutically acceptable salt thereof, is administered in an amount that is between about 150 mg and about 250 mg once a week (QW).

In some embodiments, compound 1, or a pharmaceutically acceptable salt thereof, is administered in an amount that is about 100 mg once a week (QW). In some embodiments, compound 1, or a pharmaceutically acceptable salt thereof, is administered in an amount that is about 150 mg once a week (QW). In some embodiments, compound 1, or a pharmaceutically acceptable salt thereof, is administered in an amount that is about 200 mg once a week (QW). In some embodiments, compound 1, or a pharmaceutically acceptable salt thereof, is administered in an amount that is about 250 mg once a week (QW).

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is about 25 mg, 30 mg, 40 mg, 50 mg, about 60 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments, each of the above-recited amounts may be administered QD, QW, BID, BID-QD, or BID-QW.

### Administration

Administration of compound 1, or a pharmaceutically acceptable salt thereof, and combination partners described herein are at a dosage described herein or at other dose levels and compositions determined and contemplated by a medical practitioner. In certain embodiments, compound 1, or a pharmaceutically acceptable salt thereof, is administered for prophylactic and/or therapeutic treatments. In certain therapeutic applications, compound 1, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1, or a pharmaceutically acceptable salt thereof, and combination partners described herein, are administered once a day. In some embodiments, compound 1, or a pharmaceutically acceptable salt thereof, and combination partners described herein are administered twice a day. In some embodiments, compound 1, or a pharmaceutically acceptable salt thereof, and combination partners described herein are administered three times a day.

In some embodiments, encorafenib is administered once daily. In some embodiments. encorafenib is administered twice daily. In some embodiments, encorafenib is administered three times daily.

In some embodiments, cetuximab is administered once every three weeks. In some embodiments, cetuximab is administered once every four weeks. In some embodiments, cetuximab is administered once every five weeks. In some embodiments, cetuximab is administered once every six weeks.

In some embodiments, compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, and combination partners described herein, are administered to a subject who is in a fasted state for at least 10 hours. In yet other embodiments, compound 1, or a pharmaceutically acceptable salt thereof, and combination partners described herein, are administered to a subject who is in a fasted state for at least 12 hours. In other embodiments, compound 1, or a pharmaceutically acceptable salt thereof, and combination partners described herein, are administered to a subject who has fasted overnight.

In other embodiments, compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal. 20) minutes post-meal, 30) minutes post-meal, 40) minutes post-meal, 50 minutes post-meal, 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, and combination partners described herein, are administered to a subject in a fed state 1 hour post-meal. In yet further embodiments, compound 1, or a pharmaceutically acceptable salt thereof, is administered to a subject with food.

The length of a treatment cycle depends on the treatment being given. In some embodiments. the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1, or a pharmaceutically acceptable salt thereof, and combination partners described herein are administered in 28-day cycles. In some embodiments of a method of treating cancer, compound 1, or a pharmaceutically acceptable salt thereof, and combination partners described herein, are administered for multiple 28-day cycles. In some embodiments of a method of treating cancer, compound 1, or a pharmaceutically acceptable salt thereof. and combination partners described herein, are administered for at least one 28-day cycle. In some embodiments of a method of treating cancer, compound 1, or a pharmaceutically acceptable salt thereof. and combination partners described herein, are administered for at least two 28-day cycles. In some embodiments of a method of treating cancer, compound 1, or a pharmaceutically acceptable salt thereof. and combination partners described herein, are administered for at least three 28-day cycles. In some embodiments of a method of treating cancer, compound 1, or a pharmaceutically acceptable salt thereof. and combination partners described herein, are administered for at least four 28-day cycles. In some embodiments of a method of treating cancer, compound 1, or a pharmaceutically acceptable salt thereof. and combination partners described herein, are administered for at least five 28-day cycles. In some embodiments of a method of treating cancer, compound 1, or a pharmaceutically acceptable salt thereof. and combination partners described herein, are administered for at least six 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof. is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle. In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, and combination partners described herein are administered in 35-day cycles. In some embodiments of a method of treating cancer, compound 1, or a pharmaceutically acceptable salt thereof, and combination partners described herein are administered for multiple 35-day cycles. In some embodiments of a method of treating cancer, compound 1, or a pharmaceutically acceptable salt thereof. and combination partners described herein are administered for at least one 35-day cycle. In some embodiments of a method of treating cancer, compound 1, or a pharmaceutically acceptable salt thereof. and combination partners described herein are administered for at least two 35-day cycle. In some embodiments of a method of treating cancer, compound 1, or a pharmaceutically acceptable salt thereof. and combination partners described herein are administered for at least three 35-day cycle. In some embodiments of a method of treating cancer, compound 1, or a pharmaceutically acceptable salt thereof. and combination partners described herein are administered for at least four 35-day cycle. In some embodiments of a method of treating cancer, compound 1, or a pharmaceutically acceptable salt thereof. and combination partners described herein are administered for at least five 35-day cycle. In some embodiments of a method of treating cancer, compound 1, or a pharmaceutically acceptable salt thereof. and combination partners described herein are administered for at least six 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof. is administered on days 1-28 of each 35-day cycle. In some embodiments of a method of treating cancer. compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating a cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

### Patient Selection

In some embodiments of a method of treating cancer, the subject is 18 years of age. In another embodiment, the subject is 18 years of age or older. In another embodiment, the subject is no more than 99 years of age.

In some embodiments of a method of treating cancer, the subject is willing and able to give written informed consent.

In some embodiments of a method of treating cancer, the subject has histologically or cytologically confirmed metastatic CRC harboring applicable mutations based on an analytically validated assay performed on tumor tissue in a certified testing laboratory. In some embodiments, the subject has a BRAFm V600E mutations. In some embodiments, the subject has a KRAS mutation. In some embodiments, the subject has a NRAS mutation.

In some embodiments of a method of treating cancer, the subject has histologically or cytologically confirmed advanced NSCLC harboring one or more EGFR mutations.

In some embodiments of a method of treating cancer, the subject has adequate bone marrow and organ function.

In some embodiments of a method of treating cancer, the subject has Eastern Cooperative Oncology Group (ECOG) performance status of 0 or 1.

In some embodiments of a method of treating cancer, the subject is willing to comply with all protocol-required visits, assessments, and procedures.

In some embodiments of a method of treating cancer, the subject is able to swallow oral medication.

In some embodiments of a method of treating cancer, the subject has not received prior therapy with a RAS, MEK, or ERK inhibitor.

In some embodiments of a method of treating cancer, the subject is not receiving concurrent treatment with any systemic anticancer therapy for NSCLC.

In some embodiments of a method of treating cancer, the subject has not received prior cancer immunotherapy (CIT), unless the CIT was followed by a non-CIT containing regiment prior to study enrollment.

In some embodiments of a method of treating cancer, the subject has not received an anticancer treatment within 21 days of enrollment, except for osimertinib which may be continued during the screening period.

In some embodiments of a method of treating cancer, the subject does not have a history of unacceptable toxicity to treatment with osimertinib.

In some embodiments of a method of treating cancer, the subject has not received anti-cancer therapy ≤21 days prior to first dose of the study drug or a drug combination.

In some embodiments of a method of treating cancer, the subject has not received anti-cancer therapy 4 half-lives prior to first dose of the study drug or a drug combination.

In some embodiments of a method of treating cancer, the subject has not received palliative radiation ≤7 days prior to first dose of study drug or a drug combination.

In some embodiments of a method of treating cancer, the subject does not have or has not been diagnosed with symptomatic brain metastasis or a leptomeningeal disease.

In some embodiments of a method of treating cancer, the subject does not have or has not been diagnosed with gastrointestinal conditions that may affect absorption of oral medications.

In some embodiments of a method of treating cancer, the subject does not have or has not been diagnosed with active infection requiring systemic therapy, or a history of HIV infection, hepatitis B virus, or hepatitis C virus.

In some embodiments of a method of treating cancer, the subject does not have a history of chronic inflammatory bowel disease or Crohn's disease requiring medical intervention, such as immunomodulatory, immunosuppressive medications or surgery. ≤12 months prior to first dose of study drug or a drug combination.

In some embodiments of a method of treating cancer, the subject does not have an active and clinically significant interstitial lung disease or pneumonitis.

In some embodiments of a method of treating cancer, the subject does not have an impaired cardiovascular function or clinically significant cardiovascular disease.

In some embodiments of a method of treating cancer, the subject does not have a history of thromboembolic or cerebrovascular events ≤6 months prior to first dose of study drug or a drug combination.

In some embodiments of a method of treating cancer, the subject does not have a history of a history or current evidence of retinal pigment epithelial detachment (RPED), central serous retinopathy. retinal vein occlusion (RVO), or predisposing factors to RPED or RVO.

In some embodiments of a method of treating cancer, the subject has not had major surgery within 28 days of enrollment of the study or anticipate of major surgery during study treatment.

In some embodiments of a method of treating cancer, the subject does not have known intolerance or contraindication to encorafenib, cetuximab, or palbociclib.

In some embodiments of a method of treating cancer, the subject does not have known intolerance or contraindication to osimertinib.

In some embodiments of a method of treating cancer, the subject is not pregnant or is not breastfeeding.

In some embodiments of a method of treating cancer, the subject does not have any evidence of severe or uncontrolled systemic disease or evidence of any other significant clinical disorder or laboratory finding that renders the patient inappropriate to participate in the study.

### EXAMPLES

### Example 1: In-Vivo Assay (Compound 1+Encorafenib/Cetuximab) Vehicle/Control Article

The vehicle/control article, 0.5% Methyl Cellulose & 0).1% Tween 80, or 100 mM acetic acid in deionized water with pH adjustment to 4.8-5.0, was prepared and stored under ambient conditions throughout the study period.

### Formulation of Test Article

The test article compound 1 was freshly prepared in vehicle of 0.5% Methyl Cellulose & 0.1% Tween 80 weekly and stored under ambient conditions. The combination agent, encorafenib, was freshly prepared in vehicle of 0.5% CMC and 0.5% Tween 80 weekly and stored at 2-8° C. The combination agent, cetuximab, was diluted with 1×PBS and stored at 2-8° C, before dosing.

### Animals

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign and WuXi AppTec. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign and WuXi AppTec adhered to the study protocols reviewed and approved by IACUC and applicable standard operating procedures (SOPs).

### Preparation of Xenograft Model

RKO is a human CRC tumor cell line that harbors a BRAF^{V600E} mutation. The RKO cell line was purchased from the American Type Culture Collection (ATCCR: CRL-2577™). RKO cells were cultured in medium containing EMEM plus 10% Fetal Bovine Serum (FBS) supplemented with non-essential amino acids at 37° C, in an atmosphere of 5% CO₂ in air. RKO cells in 200 μL cell suspensions containing 2×10⁶ cells mixed with 50% Matrigel were implanted into mice subcutaneously. When tumor volumes reached a mean of 200 mm³, tumor-bearing mice were randomized into different groups with 8 mice in each group and treatment started on the day of randomization.

WiDr is a human CRC tumor cell line that harbors a BRAF^{V600E} mutation. The WiDr cell line was purchased from the European Collection of Authenticated Cell Cultures (ECACC. 85111501). WiDr cells were cultured in medium containing EMEM (EBSS) plus 10% Fetal Bovine Serum (FBS). 2 mM Glutamine, and supplemented with 1% non-essential amino acids (NEAA) at 37° C, in an atmosphere of 5% CO₂ in air. WiDr cells in 200 μL cell suspensions containing 5×10⁶ cells mixed with 50% Matrigel were implanted into mice subcutaneously. When tumor volumes reached a mean of 190 mm³, tumor-bearing mice were randomized into different groups with 8 mice in each group and treatment started on the day of randomization.

### Treatment

Mice were dosed by oral administration of vehicle control solution, compound 1, encorafenib, or cetuximab in monotherapy treatment groups. Mice were dosed by oral administration of combinations including compound 1 with encorafenib and compound 1 with encorafenib plus cetuximab. The dosing volume was 5 mL/kg for each compound and interval of BID regimen was 8 hours. In the combination of compound 1 with encorafenib, compound 1 was dosed at one-hour post encorafenib dose. In the combination of compound 1 with encorafenib plus cetuximab, compound 1 was dosed at one-hour post encorafenib and cetuximab was dosed at one-hour post compound 1 QD or first BID dose. In addition to regular food and water supply, DietGel (ClearH2O, US) was added in cages where at least two mice showed >10% BWL. The study was terminated at the end of 4-week treatment or when tumor volume in vehicle control group reached 2,000 mm³.

### Results

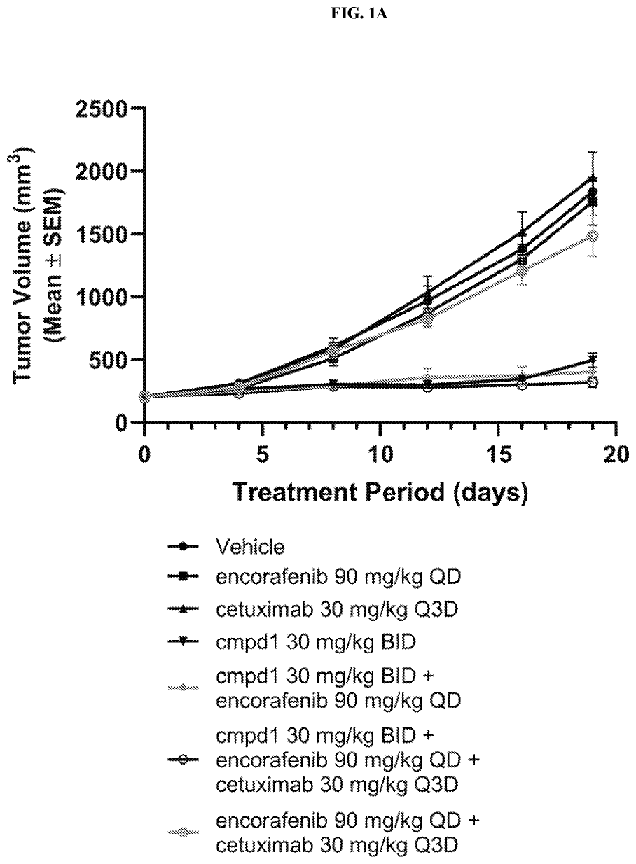

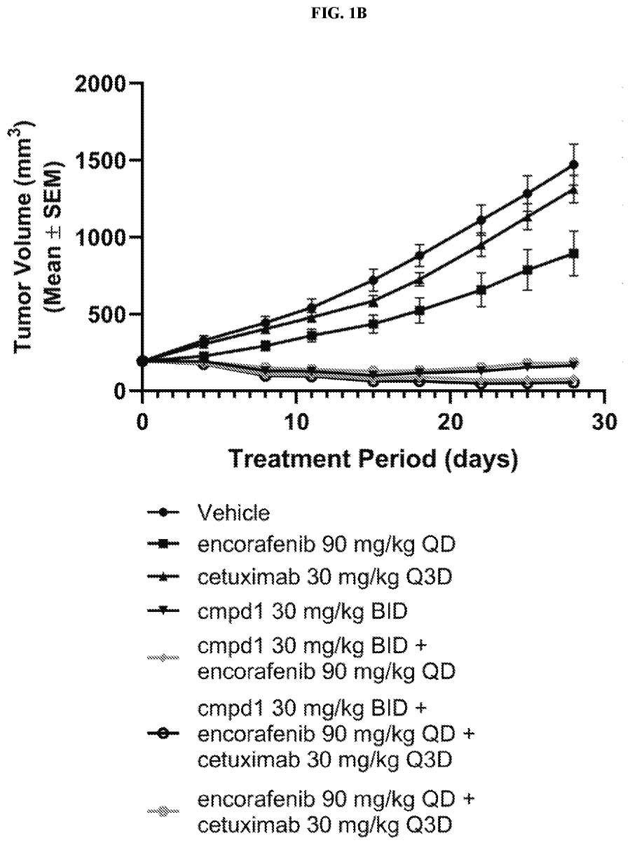

In the BRAF V600E CRC CDX model RKO, compound 1 exhibited 82% tumor growth inhibition (TGI) as a monotherapy (p-value<0.001). 88% TGI in combination with encorafenib (p-value<0.001) and 93% TGI in combination with encorafenib and cetuximab (p-value<0.001), as illustrated by the tumor growth curves of FIG. 1A.

In the BRAF V600E CRC CDX model WiDr, compound 1 exhibited 102% TGI as a monotherapy (p-value<0.001), 109% TGI in combination with encorafenib (p-value<0.001), and 111% TGI in combination with encorafenib and cetuximab (p-value<0.001), as illustrated by the tumor growth curves of FIG. 1B.

Compound 1+encorafenib and compound 1+encorafenib/cetuximab demonstrated combination benefit in vivo in encorafenib/cetuximab (EC) refractory-BRAF^{V600E} CDX models.

### Example 2: In-Vivo Assay (Compound 1+Osimertinib)

### Vehicle/Control Article

The vehicle/control article, 0.5% Methyl Cellulose & 0.1% Tween 80 or 100 mM acetic acid in deionized water with pH adjustment to 4.8-5.0, was prepared and stored under ambient conditions throughout the study period.

### Formulation of Test Article

The test article, compound 1, was freshly prepared in vehicle of 0.5% Methyl Cellulose & 0.1% Tween 80 weekly and stored under ambient conditions. The combination agent, osimertinib, was prepared weekly in vehicle of 0.5% HPMC and 0.1% Tween 80 weekly and stored under ambient conditions.

### Animals

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign and WuXi AppTec. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign and WuXi AppTec adhered to the study protocols reviewed and approved by IACUC and applicable standard operating procedures (SOPs).

### Preparation of Xenograft Model

LUN2355-214 PDX was established for pre-clinical efficacy study at GenenDesign. This PDX model was derived from a 49-year old male Chinese NSCLC patient. EGFR L858R, T790 and C797 mutations in the LUN2355-214 PDX model was confirmed by whole exome sequencing and PCR sequencing. LUN2355-128-33 PDX was established for pre-clinical efficacy study at GenenDesign. This PDX model was derived from a 49-year old male Chinese NSCLC patient. EGFR L858R and T790 mutations in the LUN2355-128-33 PDX model was confirmed by whole exome sequencing and PCR sequencing. Both LUN2355-214 and LUN2355-128-33 PDX models were treated and derived after long term treatment with osimertinib before using for the combination efficacy study of compound 1 with combination agents. PDX tumor fragments (15-30 mm³) were implanted into mice subcutaneously. When tumor volumes reached a mean of 200 mm³, tumor-bearing mice were randomized into different groups with 8 mice in each group and treatment started on the day of randomization.

### Treatment

Mice were dosed by oral administration of vehicle control solution, compound 1, or osimertinib in monotherapy treatment groups. Mice were dosed by oral administration of combinations, including compound 1 with osimertinib. The dosing volume was 5 mL/kg for each compound and interval of BID regimen was 8 hours. In the combination of compound 1 with osimertinib, compound 1 was dosed at one-hour post osimertinib dose. In addition to regular food and water supply. DietGel (ClearH2O, US) was added in cages where at least two mice showed >10% BWL. The study was terminated at the end of 4-week treatment or when tumor volume in vehicle control group reached 2,000 mm³.

### Results

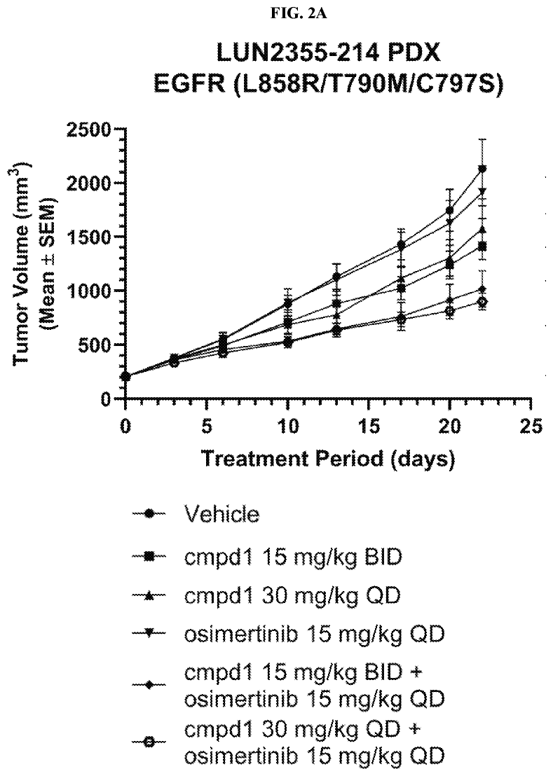

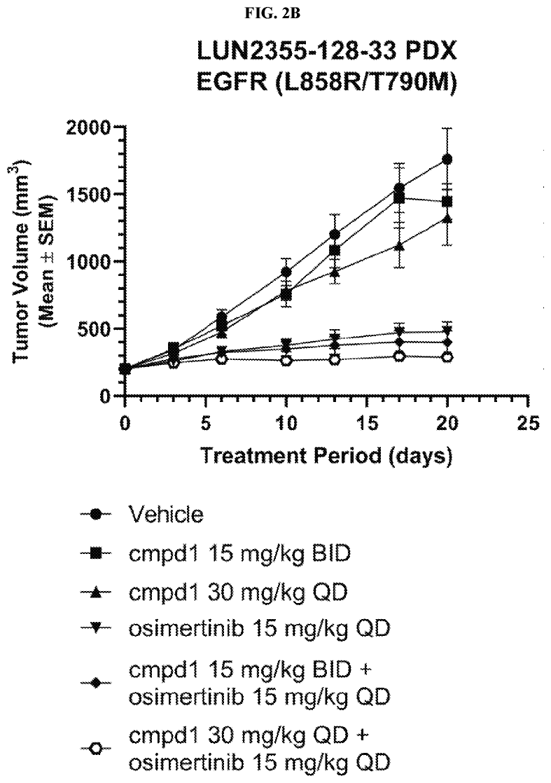

Compound 1 and osimertinib demonstrated combination benefit in vivo in osimertinib refractory EGFR mutant PDX models (see, e.g., tumor growth curves for LUN2355-214 (FIG. 2A) and LUN2355-128-33 (FIG. 2B) PDX models).

### Example 3: Phase 1b/2 Study of Compound 1, Cetuximab, and Encorafenib Targeting the MAPK Pathway in Patients with Advanced Gastrointestinal Malignancies

### Study Design

This Phase 1b/2 sub-study will evaluate the safety, clinical pharmacology, and preliminary efficacy of compound 1 administered QW in combination with encorafenib and cetuximab in patients with previously treated BRAF V600E CRC. Part 1 will determine the Recommended Dose (RD) for compound 1 once weekly (QW) in combination with encorafenib once daily (QD) and cetuximab once every two weeks (Q2W). Part 2 will further evaluate the RD from Part 1 in a larger cohort of patients. If opened, Part 3 will determine the RD for compound 1 QD in combination with encorafenib QD and cetuximab Q2W. Part 4 will further evaluate the RD from Part 3 in a larger cohort of patients. The decision to initiate Part 3 will depend on a review of data from Part 1. Only 1 RD from Part 1 or 3 will be selected for expansion in Part 2 or 4, respectively.

Up to approximately 296 patients with BRAF V600E CRC may be enrolled in this sub-study. In Parts 1 and 3, up to approximately 36 safety-evaluable patients may be enrolled in each dose-escalation part. Patients who withdraw from the study during the 28-day dose-limiting toxicity (DLT) evaluation period due to reasons not related to treatment-emergent toxicities will be replaced. In Part 2 or Part 4 (only one of these parts will be selected for expansion), up to approximately 200 efficacy—evaluable patients (receiving at least 1 dose of compound 1 and having at least 1 post-dose tumor assessment) may be enrolled in the applicable part, with up to approximately 100 efficacy-evaluable patients in each cohort. Assuming approximately 10% of dosed patients withdrawing from the study prior to the first post-dose tumor assessment, dose-expansion cohorts may enroll and dose up to approximately 224 patients.

### Parts 1 and 3 (Dose Escalation)

Parts 1 and 3 will evaluate escalating dose levels of compound 1 in combination with encorafenib and cetuximab in patients with previously treated BRAF V600E CRC, regardless of prior BRAF inhibitor and EGFR inhibitor treatment, using a rolling six design.

In Part A1, compound 1 will be evaluated at multiple QW dose levels between 150 and 250 mg (inclusive), such as 150 mg QW, 200 mg QW, and 250 mg QW, with 150 mg QW as the starting dose, in combination with encorafenib at 300 mg QD and cetuximab at 500 mg/m² Q2W.

In Part A2, compound 1 will be evaluated at multiple BID-QW dose levels between 75 mg and 125 mg (inclusive), such as 75 mg BID-QW, 100 mg BID-QW, and 125 mg BID-QW, with 75 mg BID-QW as the starting dose, in combination with encorafenib at 300 mg QD and cetuximab at 500 mg/m² Q2W.

In Part A3, compound 1 will be evaluated at multiple QD dose levels between 20 and 40 mg (inclusive), such as 20 mg QD, 30 mg QD, and 40 mg QD, with 20 mg QD as the starting dose, in combination with encorafenib at 300 mg QD and cetuximab at 500 mg/m² Q2W.

After the MTD is identified, up to approximately 15 patients per dose level may be enrolled at the MTD level and 1 dose level below MTD to further evaluate safety, tolerability, and PK/PD. For example, if 200 mg QW is the MTD for Part 1, then the 200 mg QW cohort and 150 QW cohort may each enroll up to approximately 15 patients total (inclusive of patients who have already been enrolled under the rolling six design). The data will be used by the Sponsor, in consultation with the SRC and investigators, to select the RD in combination with encorafenib and cetuximab. The Sponsor will inform the investigators of the RD selection.

### Parts 2 and 4 (Dose Expansion)

In Part 2 or Part 4 (only one of these parts will be selected for expansion), the efficacy of compound 1 plus encorafenib and cetuximab in EC-naïve or previously treated patients with BRAF V600E CRC who either have or have not received prior BRAF inhibitor and EGFR inhibitor therapy will be evaluated in multiple steps. Interim analyses will be conducted for each expansion cohort in Part 2 or Part 4 to guide potential early stopping of enrollment when there is no evidence of activity.

In Part 2 or Part 4, eligible patients with BRAF V600E CRC will be enrolled in 1 of 2 Cohorts, 2A/4A (patients that have not received prior treatment with BRAF and EGFR inhibitors) or 2B/4B (patients who have received prior treatment with BRAF and EGFR inhibitors).

For Cohort 2A or 4A, an interim analysis will be conducted in the chosen cohort once approximately 20 evaluable patients enrolled have completed at least 1 post-dose tumor assessment. If the interim analysis of ORR suggests that anti-tumor activity is below a threshold for that patient population (e.g., if less than 2 responders are observed in the first 20 patients for Part 2A), then enrollment in that cohort will be stopped. Otherwise up to approximately 10 additional evaluable patients will be enrolled in that cohort to allow for sufficient evaluation. Additionally, up to 70 patients will be enrolled in that cohort for the primary efficacy analyses. Safety, PK, and PD data will support the efficacy analyses and the decision to continue enrollment.

For Cohort 2B or 4B, an interim analysis will be conducted in the chosen cohort once approximately 30 efficacy-evaluable patients dosed have completed at least 1 post-dose tumor assessment. If the interim analysis of ORR suggests that anti-tumor activity is below a threshold for that patient population (e.g., if no responders are observed in the first 30 evaluable patients for Part 2B), then enrollment in that cohort will be stopped. Otherwise, up to approximately 70 additional evaluable patients will be enrolled in that cohort for the primary efficacy evaluation. Safety, PK, and PD data will support the efficacy analyses and the decision to continue enrollment.

### Dosing Schedule

Compound 1 + Encorafenib in BRAF V600E CRC

| | | |
|---|---|---|
| A1: Compound 1 QW^{a} + EC^{d} | A2: Compound 1 BID-QW^{b} + | A3: Compound 1 QD^{c} + EC^{d} |
| in EC-naïve or -treated BRAF | EC^{d} in EC-naïve or -treated | in EC-naïve or -treated BRAF |
| V600E CRC | BRAF V600E CRC | V600E CRC |

Dose Escalation

| | | |
|---|---|---|
| Compound 1 150 mg QW + EC | Compound 1 75 mg BID-QW + EC | Compound 1 20 mg QD + EC |
| Compound 1 200 mg QW + EC | Compound 1 100 mg BID-QW + EC | Compound 1 30 mg QD + EC |
| Compound 1 250 mg QW + EC | Compound 1 125 mg BID-QW + EC | Compound 1 40 mg QD + EC |

Dose Expansion

| | | |
|---|---|---|
| Compound 1 QW + EC (RD) | Compound 1 75 mg BID-QW + EC | Compound 1 20 mg QD + EC |

^{a}Compound 1 QW—Compound 1oral administration once a week;
^{b}Compound 1 BID-QW—Compound 1oral administration twice a day on a single day each week;
^{c}Compound 1 QD—Compound oral administration once a day;
^{d}EC: Encorafenib 300 mg oral daily + Cetuximab 500 mg/m² intravenous infusion once every 2 week.

### Inclusion Criteria

Age≥18 years.

Willing and able to give written informed consent.

Have histologically or cytologically confirmed metastatic CRC harboring applicable mutation(s) (e.g., BRAFm V600E; KRAS or NRAS mutations) based on an analytically validated assay performed on tumor tissue in a certified testing laboratory.

Measurable disease per Response Evaluation Criteria in Solid Tumors (RECIST) v1.1.

Adequate bone marrow and organ function.

Have ECOG performance status of 0 or 1.

Willing to comply with all protocol-required visits, assessments, and procedures.

Able to swallow oral medication.

### Exclusion Criteria

Prior therapy with a RAS, MEK, or ERK inhibitor. Depending on which treatment arm the patient is assigned, other therapies could also be prohibitive.

Anti-cancer therapy≤21 days or 4 half-lives prior to first dose of study drug, whichever is shorter.

Palliative radiation≤7 days prior to first dose of study drug.

Symptomatic brain metastasis or leptomeningeal disease.

Gastrointestinal conditions that may affect absorption of oral medications.

Active infection requiring systemic therapy, or history of HIV infection, hepatitis B virus, or hepatitis C virus.

History of chronic inflammatory bowel disease or Crohn's disease requiring medical

intervention (immunomodulatory or immunosuppressive medications or surgery)≤12 months prior to first study drug dose.

Active, clinically significant interstitial lung disease or pneumonitis.

Impaired cardiovascular function or clinically significant cardiovascular disease.

History of thromboembolic or cerebrovascular events≤6 months prior to first dose.

Major surgery within 28 days of enrollment or anticipate of major surgery during study treatment.

Known intolerance or contraindication to encorafenib, cetuximab, or Palbociclib.

Pregnant or breastfeeding women.

Any evidence of severe or uncontrolled systemic disease or evidence of any other significant clinical disorder or laboratory finding that renders the patient inappropriate to participate in the study.

### Example 4: A Phase 1b/2, Open-Label, Multicenter Study of Compound 1 in Combination with Osimertinib in Patients with Advanced Non-Small-Cell Lung Cancer

Detailed Description: This is a Phase 1b/2, open-label, multicenter clinical study evaluating Compound 1 in combination with osimertinib in study participants with advanced NSCLC. This study will serve as a platform study, allowing for evaluation of safety/tolerability and efficacy of Compound 1 in combination with other cancer therapies. The study will initially commence with dose escalation of Compound 1, administered at either the doses of 150 mg once weekly (QW), 200 mg QW, and 250 mg QW respectively, or a dosing schedule of twice a day once weekly (BID-QW), in combination with Osimertinib, administered at 80 mg once daily (QD), in study participants with advanced NSCLC harboring an epidermal growth factor receptor-sensitizing mutation (EGFRm). Dose expansion will follow and will test Compound 1 administered at the QW recommended dose (RD), or BID-QW RD identified from dose escalation, in combination with osimertinib in study participants with EGFRm NSCLC.

### Arms and Interventions

| Arms | Assigned Interventions |
|---|---|
| Experimental: Dose Escalation (Part 1): | Drug: Compound 1 |
| Compound 1 QW plus osimertinib | Compound 1 will be administered orally as |
| Compound 1 will be orally administered QW in | specified in Arm description. |
| combination with osimertinib to study | Drug: Osimertinib 80 mg orally once daily |
| participants with advanced EGFRm NSCLC in | |
| sequential ascending doses until unacceptable | |
| toxicity, disease progression, or withdrawal of | |
| consent. | |
| Experimental: Dose Expansion (Part 2): | Drug: Compound 1 |
| Compound 1 QW plus osimertinib | Compound 1 will be administered orally as |
| Compound 1 will be orally administered at the | specified in Arm description. |
| recommended dose (as determined from Part 1) in | Drug: Osimertinib 80 mg orally once daily |
| combination with osimertinib to study | |
| participants with advanced EGFRm NSCLC. | |

### Dosing Schedule

Compound 1 QW + Osimertinib in EGFRm NSCLC

| Dose Escalation in EGFRm NSCLC | | Dose Expansion in 2 L EGFRm NSCLC | |
|---|---|---|---|
| Part A1 | Part A3 | Part A2 | Part A4 |
| Compound 1 150 mg | Compound 1 BID-QW | MAPK Alterations | MAPK Alterations |
| QW + Osimertinib | RD + Osimertinib | Compound 1 QW RD + | Compound 1 BID-QW |
| | | Osimertinib | RD + Osimertinib |
| Compound 1 200 mg | Compound 1 BID-QW | RTK Alterations | RTK Alterations |
| QW + Osimertinib | RD-1 + Osimertinib | Compound 1 QW RD + | Compound 1 BID-QW |
| | | Osimertinib | RD + Osimertinib |
| Compound 1 250 mg | Compound 1 BID-QW | | |
| QW + Osimertinib | RD-2 + Osimertinib | | |

Compound 1 BID-QW + Osimertinib in EGFRm NSCLC

| Dose Escalation of in | Dose Expansion of in | Dose Escalation of in | Dose Expansion of in |
|---|---|---|---|
| EGFRm NSCLC | 2 L EGFRm NSCLC | EGFRm NSCLC | 2 L EGFRm NSCLC |
| Compound 1 150 mg | MAPK Alterations | Compound 1 BID-QW | MAPK Alterations |
| QW + Osimertinib | Compound 1 QW RD + | RD + Osimertinib | Compound 1 BID-QW |
| | Osimertinib | | RD + Osimertinib |
| Compound 1 200 mg | RTK Alterations | Compound 1 BID-QW | RTK Alterations |
| QW + Osimertinib | Compound 1 QW RD + | RD-1 + Osimertinib | Compound 1 BID-QW |
| | Osimertinib | | RD + Osimertinib |
| Compound 1 250 mg | | Compound 1 BID-QW | |
| QW + Osimertinib | | RD-2 + Osimertinib | |

### Outcome Measures

### Primary Outcome Measure

1. Dose Limiting Toxicities (DLT). Based on adverse events observed [Time Frame: Study Day 1 up to Day 29]
2. Maximum Tolerated Dose (MTD). Based on adverse events observed [Time Frame: Study Day 1 up to Day 29]
3. Recommended Dose (RD). Based on adverse events observed [Time Frame: Study Day 1 up to Day 29]
4. Adverse Events. Incidence and severity of treatment-emergent AEs and serious AEs [Time Frame: Assessed up to 24 months from time of first dose]

### Secondary Outcome Measure

5. Plasma concentration (Cmax). Maximum plasma concentration of compound 1 and other cancer therapies [Time Frame: Study Day 1 up to Day 29]
6. Time to achieve Cmax (Tmax). Time to achieve maximum plasma concentration of compound 1 and other cancer therapies [Time Frame: Study Day 1 up to Day 29]
7. Area under the curve. Area under the plasma concentration-time curve of compound 1 and other cancer therapies [Time Frame: Study Day 1 up to Day 29]
8. Half-life. Half-life of compound 1 and other cancer therapies [Time Frame: Study Day 1 up to Day 29]
9. Objective Response Rate (ORR). Based on assessment of radiographic imaging per RECIST version 1. 1 [Time Frame: Assessed up to 24 months from time of first dose]
10. Duration of Response (DOR). Based on assessment of radiographic imaging per RECIST version 1.1 [Time Frame: Assessed up to 24 months from time of first dose]

### Criteria: Inclusion Criteria

Age≥18 years.

Willing and able to give written informed consent.

Have histologically or cytologically confirmed advanced NSCLC harboring EGFR mutation(s) sensitive to EGFR inhibitors at initial diagnosis per local approved label.

Measurable disease per Response Evaluation Criteria in Solid Tumors (RECIST) v1.1.

Adequate bone marrow and organ function.

Have ECOG performance status of 0 or 1.

Willing to comply with all protocol-required visits, assessments, and procedures.

Able to swallow oral medication.

### Exclusion Criteria

Concurrent treatment with any systemic anticancer therapy for NSCLC, including any approved or investigational agent.

Prior therapy with a RAS, RAF, MEK, or ERK inhibitor.

Prior cancer immunotherapy (CIT) (e.g., immune checkpoint inhibitors), unless the CIT was followed by a non-CIT containing regimen prior to study enrollment

Anticancer treatment within 21 days of enrollment, except for osimertinib which may be continued during the screening period.

Palliative radiotherapy within 7 days of enrollment.

History of unacceptable toxicity to treatment with osimertinib.

Major surgery within the 28 days of enrollment.

Unresolved toxicities from prior systemic therapy greater than NCI CTCAE

grade 1 at time of enrollment, except alopecia and grade 2 neuropathy due to prior chemotherapy.

Any evidence of severe or uncontrolled systemic disease or evidence of any other significant clinical disorder or laboratory finding that renders the patient inappropriate to participate in the study.

Impaired cardiovascular function or clinically significant cardiovascular disease.

History or current evidence of retinal pigment epithelial detachment (RPED), central serous retinopathy, retinal vein occlusion (RVO), or predisposing factors to RPED or RVO.

Pregnant or breastfeeding women.

Contraindication to osimertinib use as per local label.

### Example 5: In-Vivo Studies of Compound 1 Alone, and in Combination with Encorafenib in Encorafenib Refractory-BRAF V600E CR0004 PDX Models

The vehicle/control article, 100 mM acetic acid in deionized water, with pH adjustment to 4.8-5.0, was prepared and stored under ambient conditions throughout the 28-day administration in mice.

The test article Compound 1 was prepared weekly in vehicle of 0.5% CMC and 0.5% Tween 80) weekly and stored under ambient conditions. Encorafenib was prepared in vehicle of 0.5% CMC and 0.5% Tween 80 weekly and stored at 2-8° C.

Female Balb/c nude mice were purchased from the Beijing Anikeeper Biotech Co., Ltd (Beijing, China). Mice were between 9-11 weeks of age at the time of implantation. Mice were hosted at a special pathogen-free (SPF) environment of the vivarium facility and acclimated to their new environment for at least 3 days prior to initiation of any experiments according to IACUC protocol. All procedures related to animal handling, care, and treatment in this study were performed according to guidelines approved by the Institutional Animal Care and Use Committee (IACUC) of Crown Bioscience (Beijing, China). During the study, the care and use of animals were conducted in accordance with the regulations of the Association for Assessment and Accreditation of Laboratory Animal Care (AAALAC). In addition, all portions of this study performed at Crown Bioscience (Beijing. China) adhered to the study protocols approved by the study director and applicable standard operating procedures (SOPs).

### Preparation of PDX

CR0004 PDX model was established for preclinical efficacy study at CrownBio. This PDX model was derived from a 44-year-old female CRC patient. A BRAF V600E mutation in this model was confirmed by exome sequencing. Mouse skin was cleaned with appropriate surgical scrub and iodophor over the right flank. Tumor fragments (2-3 mm in diameter) harvested from the PDX model were implanted subcutaneously in the right flanks of female Balb/c nude mice using a 18 g trocar needle. When mean tumor sizes reached 141 mm³ (range of 114-185 mm³), tumor-bearing mice were randomly divided into study groups with 8 mice per group.

### Treatment

Treatment started on the day of randomization. The treatment start day was denoted as treatment day 0. Mice were dosed by administration of vehicle control solution, Compound 1 at 30) mg/kg/dose BID, p.o., and encorafenib at 90 mg/kg QD, p.o. as monotherapies. An additional group received combination treatments of Compound 1 at 30 mg/kg/dose BID with encorafenib at 90 mg/kg QD. The dosing volume for each compound was 5 mL/kg and interval of BID regimen was 8 hours. In the combination group of Compound I with encorafenib, encorafenib was dosed at one hour after the first Compound 1 BID dose. The study was terminated on treatment day 28 as defined in the study protocol.

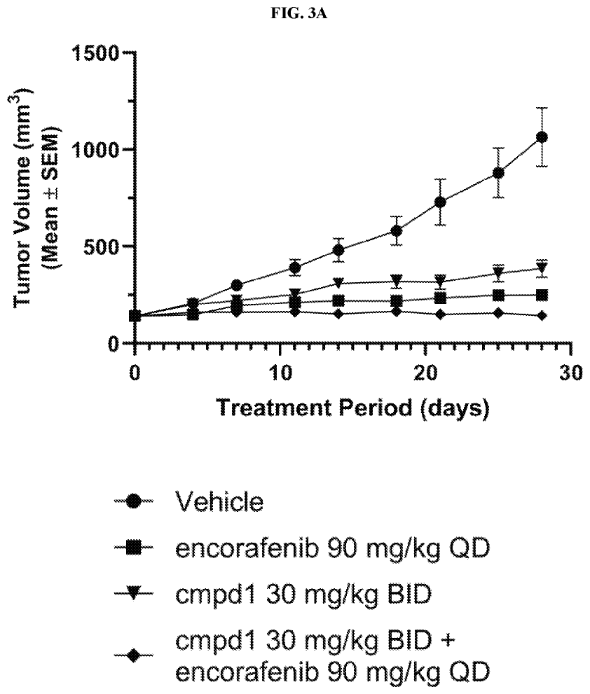

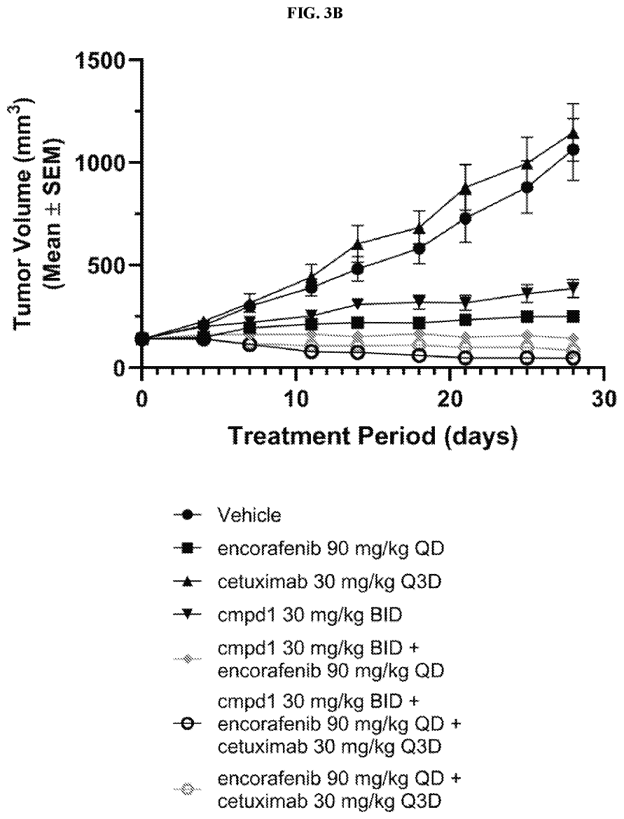

As illustrated by FIG. 3A, Compound 1 and encorafenib demonstrated combination benefit in vivo in encorafenib refractory-BRAF V600E CR0004 PDX Models.

### Example 6: In-Vivo Studies of Compound 1 Alone, and in Combination with Encorafenib and Cetuximab in Encorafenib+Cetuximab Refractory-BRAF V600E CR0004 PDX Models

The vehicle/control article, 100 mM acetic acid in deionized water, with pH adjustment to 4.8-5.0, was prepared and stored under ambient conditions throughout the 28-day administration in mice.

The test article Compound 1 was prepared weekly in vehicle of 0.5% CMC and 0.5% Tween 80 weekly and stored under ambient conditions. Encorafenib was prepared in vehicle of 0.5% CMC and 0.5% Tween 80 weekly and stored at 2-8° C.

Female Balb/c nude mice were purchased from the Beijing Anikeeper Biotech Co., Ltd (Beijing. China). Mice were between 9-11 weeks of age at the time of implantation. Mice were hosted at a special pathogen-free (SPF) environment of the vivarium facility and acclimated to their new environment for at least 3 days prior to initiation of any experiments according to IACUC protocol. All procedures related to animal handling, care, and treatment in this study were performed according to guidelines approved by the Institutional Animal Care and Use Committee (IACUC) of Crown Bioscience (Beijing. China). During the study, the care and use of animals were conducted in accordance with the regulations of the Association for Assessment and Accreditation of Laboratory Animal Care (AAALAC). In addition, all portions of this study performed at Crown Bioscience (Beijing, China) adhered to the study protocols approved by the study director and applicable standard operating procedures (SOPs).

### Preparation of PDX

CR0004 PDX model was established for preclinical efficacy study at CrownBio. This PDX model was derived from a 44-year-old female CRC patient. A BRAF^{V600E} mutation in this model was confirmed by exome sequencing. Mouse skin was cleaned with appropriate surgical scrub and iodophor over the right flank. Tumor fragments (2-3 mm in diameter) harvested from the PDX model were implanted subcutaneously in the right flanks of female Balb/c nude mice using a 18 g trocar needle. When mean tumor sizes reached 141 mm³ (range of 114-185 mm³), tumor-bearing mice were randomly divided into study groups with 8 mice per group.

### Treatment

Treatment started on the day of randomization. The treatment start day was denoted as treatment day 0. Mice were dosed by administration of vehicle control solution. Compound 1 at 30 mg/kg/dose BID, p.o., encorafenib at 90 mg/kg QD, p.o., and cetuximab at 30 mg/kg Q3D, i.p, as monotherapies. Three additional groups received combination treatments, the first group dosing Compound 1 at 30 mg/kg/dose BID with encorafenib at 90 mg/kg QD, the second group dosing Compound 1 at 30 mg/kg/dose BID with encorafenib at 90 mg/kg QD+cetuximab at 30 mg/kg Q3D, and the third group dosing encorafenib at 90 mg/kg QD with cetuximab at 30 mg/kg Q3D. The dosing volume for each compound was 5 mL/kg except for cetuximab (10 mL/kg) and interval of BID regimen was 8 hours. In the combination group of Compound I with encorafenib, encorafenib was dosed at one hour after the first Compound 1 BID dose. In the triple combination group of Compound 1 with encorafenib and cetuximab. Compound 1 BID was dosed first, encorafenib was dosed one hour later, and cetuximab was dosed one hour after encorafenib. In the combination group of encorafenib with cetuximab, encorafenib was dosed first and cetuximab was dosed one hour later. In addition to regular food and water supply. DietGel was added in cages where at least two mice started showing BWL >10% in a treatment group. Per this practice, mice in the triple combination (i.e., Compound 1 at 30) mg/kg/dose BID+encorafenib at 90 mg/kg QD+cetuximab at 30 mg/kg Q3D) were supplied with DietGel food starting on treatment day 21 and continuing through the remaining study period. The study was terminated on treatment day 28 as defined in the study protocol.

As illustrated by FIG. 3B. Compound 1 in combination with encorafenib and cetuximab demonstrated combination benefit in vivo in encorafenib+cetuximab (EC) refractory-BRAF V600E CR0004 PDX models.

### Example 7: In-vivo Studies of Compound 1 Alone, and in Combination with Encorafenib in Encorafenib Refractory-BRAF V600E CRR1011 PDX Models

The vehicle/control article of Compound 1. 0.5% Methyl Cellulose (MC) & 0.1% Tween 80 in deionized water, was prepared and stored under ambient conditions throughout the 23-day administration in mice.

The test article Compound 1 was freshly prepared in vehicle of 0.5% Methyl Cellulose (MC) & 0.1% Tween 80 solution weekly and stored under ambient conditions. Encorafenib was freshly prepared in vehicle of 0.5% CMC and 0.5% Tween 80 weekly and stored at 2-8° C.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of Xenograft Model

The PDX CRC1011 model was established for nonclinical efficacy studies at Genen Design (Shanghai, China). The PDX model was derived from a 63-year-old male Chinese CRC patient. The BRAF^{V600E} mutation in the PDX model CRC1011 was confirmed by whole exome sequencing and PCR sequencing. Tumor fragments harvested from the PDX model were implanted subcutaneously in the right flanks of female Balb/c nude mice. Mice were anesthetized with isoflurane, and anesthesia was maintained throughout the implantation procedure. The right flank of the mouse was sterilized with appropriate surgical scrub and alcohol, and aseptic surgical procedures were used. A small skin incision was made using the sharp end of the trochar and a 1.5 cm subcutaneous pocket along the right lateral chest wall was formed by blunt dissection with the stylet of a 10-12 g trocar needle. Tumor fragments (15-30 mm³) were placed into the trocar needle and advanced into the subcutaneous pocket in the right flank. The trocar incision was closed with suture or a wound clip and was removed one week after closure. When tumor sizes reached a mean of 200 mm³ (range of 144-269 mm³) in volume, tumor-bearing mice were randomly divided into study groups with 8 mice in each group. The randomization date was denoted as treatment day 0.

### Treatment

Treatment started on the day after randomization. The treatment start day was denoted as treatment day 0. Mice were dosed by administration of vehicle control solution. Compound 1 at 30 mg/kg/dose BID, p.o., and encorafenib at 90 mg/kg QD, p.o. as monotherapies. An additional group received combination treatment of Compound 1 at 30 mg/kg/dose BID with encorafenib at 90 mg/kg QD. The dosing volumes for Compound 1 and encorafenib were 5 mL/kg and interval of BID regimen was 8 hours. In the combination group of Compound 1 with encorafenib, encorafenib was dosed first and the first dose of Compound 1 BID was given one hour later. In addition to regular food and water supply. DietGel was added in cages where at least two mice started showing >10% BWL. Per this practice. mice in the Compound 1 at 30 mg/kg/dose BID monotherapy group and the Compound 1 at 30 mg/kg/dose BID with encorafenib at 90 mg/kg QD combination group were supplied with DietGel food starting on treatment day 12, 12, and 9, respectively, and continuing through the remaining study period. The study was terminated on treatment day 23, which was earlier than the original termination day (day 28) as defined in the study protocol due to rapid tumor growth in the vehicle group. Two out of eight tumors in the vehicle control group exceeded the tumor volume threshold per IACUC protocol (2,000 mm³) on treatment day 23.

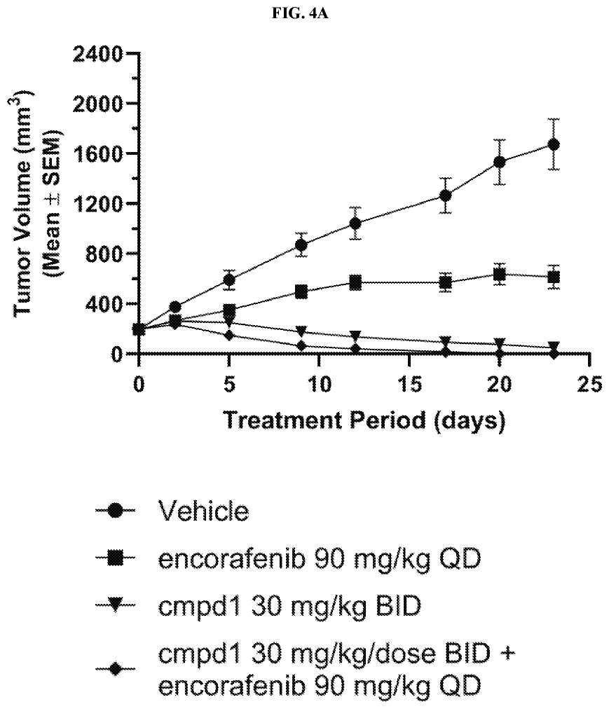

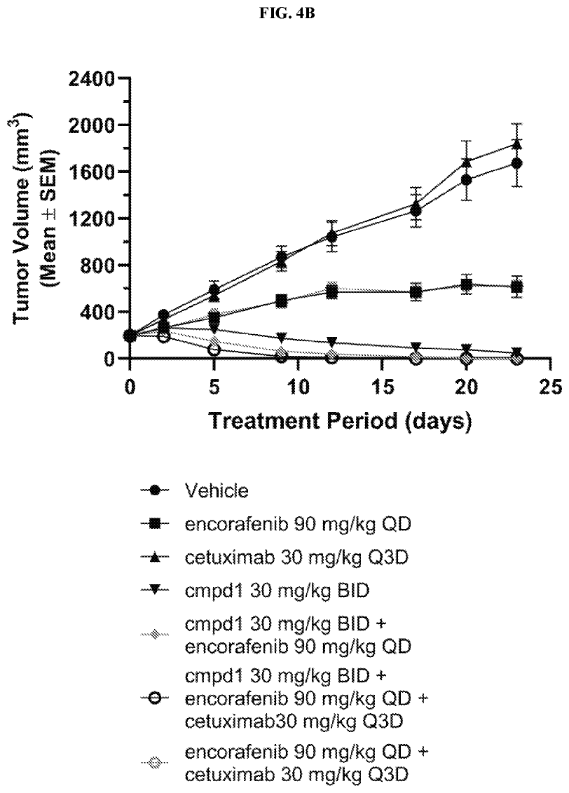

As illustrated by FIG. 4A. Compound 1 and encorafenib demonstrated combination benefit in vivo in encorafenib refractory-BRAF V600E CRC1011 PDX Models.

### Example 8: In-Vivo Studies of Compound 1 Alone, and in Combination with Encorafenib and Cetuximab in Encorafenib+Cetuximab Refractory-BRAF V600E CRC1011 PDX Models

The test article. Compound 1, was manufactured by WuXi STA in Shanghai, China. The combination agent, encorafenib, was purchased from MedChemExpress. Cetuximab was purchase from Merck KGaA in China (batch #G002VX).

The vehicle/control article of Compound 1. 0.5% Methyl Cellulose (MC) & 0.1% Tween 80 in deionized water, was prepared and stored under ambient conditions throughout the 23-day administration in mice.

The test article Compound 1 was freshly prepared in vehicle of 0.5% Methyl Cellulose (MC) & 0.1% Tween 80 solution weekly and stored under ambient conditions. Encorafenib was freshly prepared in vehicle of 0.5% CMC and 0.5% Tween 80 weekly and stored at 2-8° C. Cetuximab (5 mg/mL) was diluted with saline to 3 mg/mL before each dosing.

Female Balb/c nude mice were purchased from the Beijing Vital River Laboratory Animal Technology Co., Ltd. Mice were hosted in a special pathogen-free (SPF) environment of the vivarium facility and acclimated to their new environment for at least 3 days prior to initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of PDX

The PDX CRC1011 model was established for nonclinical efficacy studies at GenenDesign (Shanghai, China). The PDX model was derived from a 63-year-old male Chinese CRC patient. The BRAF^{V600E} mutation in the PDX model CRC1011 was confirmed by whole exome sequencing and PCR sequencing. Tumor fragments harvested from the PDX model were implanted subcutaneously in the right flanks of female Balb/c nude mice. Mice were anesthetized with isoflurane, and anesthesia was maintained throughout the implantation procedure. The right flank of the mouse was sterilized with appropriate surgical scrub and alcohol, and aseptic surgical procedures were used. A small skin incision was made using the sharp end of the trochar and a 1.5 cm subcutaneous pocket along the right lateral chest wall was formed by blunt dissection with the stylet of a 10-12 g trocar needle. Tumor fragments (15-30 mm³) were placed into the trocar needle and advanced into the subcutaneous pocket in the right flank. The trocar incision was closed with suture or a wound clip and was removed one week after closure. When tumor sizes reached a mean of 200 mm³ (range of 144-269 mm³) in volume, tumor-bearing mice were randomly divided into study groups with 8 mice in each group. The randomization date was denoted as treatment day 0.

### Treatment

Treatment started on the day after randomization. The treatment start day was denoted as treatment day 0. Mice were dosed by administration of vehicle control solution. Compound 1 at 30 mg/kg/dose BID, p.o., encorafenib at 90 mg/kg QD, p.o., and cetuximab at 30 mg/kg Q3D, i.p, as monotherapies. Three additional groups received combination treatments, the first group dosing Compound 1 at 30 mg/kg/dose BID with encorafenib at 90 mg/kg QD, the second group dosing Compound 1 at 30 mg/kg/dose BID with encorafenib at 90 mg/kg QD+cetuximab at 30 mg/kg Q3D, and the third group dosing encorafenib at 90 mg/kg QD with cetuximab at 30 mg/kg Q3D. The dosing volumes for Compound 1, encorafenib, and cetuximab were 5 mL/kg and interval of BID regimen was 8 hours. In the combination group of Compound 1 with encorafenib, encorafenib was dosed first and the first dose of Compound1 BID was given one hour later. In the combination group of Compound 1 with encorafenib and cetuximab, encorafenib was dosed first, the first dose of Compound 1 BID was given one hour later, and cetuximab was dosed one hour after Compound 1 first BID dose. In the combination group of encorafenib with cetuximab, encorafenib was dosed first and cetuximab was dosed one hour later. In addition to regular food and water supply, DietGel was added in cages where at least two mice started showing >10% BWL. Per this practice, mice in the Compound 1 at 30 mg/kg/dose BID monotherapy group, the Compound 1 at 30 mg/kg/dose BID with encorafenib at 90 mg/kg QD combination group and the Compound 1 at 30 mg/kg/dose BID with encorafenib at 90 mg/kg QD+cetuximab at 30 mg/kg Q3D combination group were supplied with DietGel food starting on treatment day 12, 12, and 9, respectively, and continuing through the remaining study period The study was terminated on treatment day 23, which was earlier than the original termination day (day 28) as defined in the study protocol due to rapid tumor growth in the vehicle group. Two out of eight tumors in the vehicle control group exceeded the tumor volume threshold per IACUC protocol (2,000 mm³) on treatment day 23.

As illustrated by FIG. 4B, Compound 1 in combination with encorafenib and cetuximab demonstrated combination benefit in vivo in encorafenib+cetuximab (EC) refractory-BRAF V600E CRC1011 PDX models.

1. A method of treating cancer in a subject in need thereof, the method comprising: administering to the subject in need thereof a therapeutically effective amount of
(i) compound 1: 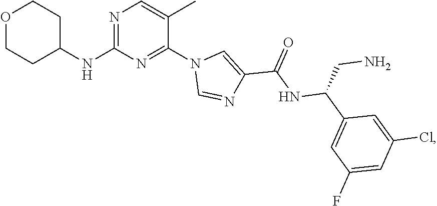 [SMILES: Cc1cnc(NC2CCOCC2)nc1-n1cnc(C(=O)N[C@H](CN)c2cc(F)cc(Cl)c2)c1]
or a pharmaceutically acceptable salt thereof, and
(ii) an EGFR inhibitor.

2. The method of claim 1, wherein the EGFR inhibitor is a small molecule EGFR inhibitor.

3. The method of claim 1, wherein the EGFR inhibitor is afatinib, amivantamab, canertinib, cetuximab, dacomitinib, daphnetin, erlotinib, gefitinib, icotinib, lapatinib, lazertinib, lifirafenib, mirzotamab clezutoclax, mobocertinib, nazartinib, necitumumab, neratinib, osimertinib, panitumamab, pelitinib, poziotinib, tivozanib, rociletinib, sapitinib, vandetanib, or varlitinib.

4. The method of any one of claims 1 to 3, wherein the EGFR inhibitor is osimertinib.

5. The method of claim 4, wherein osimertinib is administered in an amount that is about 80 mg/day.

6. The method of claim 1, wherein the EGFR inhibitor is cetuximab.

7. The method of claim 6, wherein cetuximab is initially administered at 400 mg/m² over 120 minutes followed by 250 mg/m² over 60 minutes once a week.

8. The method of claim 6, wherein cetuximab is administered at 500 mg/m² once every two weeks.

9. The method of claim 6, wherein cetuximab is administered at 400 mg/m² once every two weeks or 300 mg/m² once every two weeks.

10. The method of any one of claims 1-9, wherein the method further comprises administering a BRAF inhibitor.

11. The method of claim 10, wherein the BRAF inhibitor is dabrafenib, encorafenib, regorafenib, sorafenib, or vemurafenib.

12. The method of claim 10, wherein the BRAF inhibitor is encorafenib.

13. The method of claim 12, wherein encorafenib is administered in an amount that is between about 100 mg/day and about 500 mg/day.

14. The method of claim 12, wherein encorafenib is administered in an amount that is about 450 mg/day.

15. The method of claim 12, wherein encorafenib is administered in an amount that is about 300 mg/day.

16. The method of claim 12, wherein encorafenib is administered in an amount that is about 225 mg/day

17. The method of claim 12, wherein encorafenib is administered in an amount that is about 150 mg/day.

18. The method of claim 10, wherein the BRAF inhibitor is dabrafenib.

19. The method of claim 18, wherein dabrafenib is administered in an amount that is about 150 mg.

20. A method of treating cancer in a subject in need thereof, the method comprising: administering to the subject in need thereof a therapeutically effective amount of 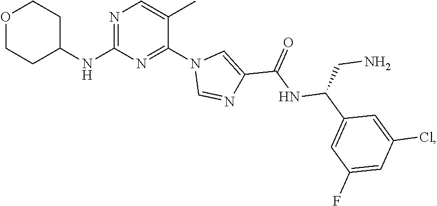 [SMILES: Cc1cnc(NC2CCOCC2)nc1-n1cnc(C(=O)N[C@H](CN)c2cc(F)cc(Cl)c2)c1]
(i) compound 1:
or a pharmaceutically
acceptable salt thereof,
(ii) an EGFR inhibitor; and
(iii) a BRAF inhibitor

21. The method of claim 20, wherein the EGFR inhibitor is a small molecule inhibitor.

22. The method of claim 20, wherein the EGFR inhibitor is afatinib, amivantamab, cetuximab, dacomitinib, erlotinib, gefitinib, lapatinib, lazertinib, lifirafenib, mirzotamab clezutoclax, mobocertinib, nazartinib, necitumumab, neratinib, osimertinib, or vandetanib.

23. The method of any one of claims 20 to 22, wherein the EGFR inhibitor is osimertinib

24. The method of claim 23, wherein osimertinib is administered in an amount that is about 80 mg/day.

25. The method of claim 22, wherein the EGFR inhibitor is cetuximab.

26. The method of claim 25, wherein cetuximab is initially administered at 400 mg/m² over 120 minutes followed by 250 mg/m² over 60 minutes once a week.

27. The method of claim 25, wherein cetuximab is administered at 500 mg/m² once every two weeks.

28. The method of claim 25, wherein cetuximab is administered at 400 mg/m² once every two weeks or 300 mg/m² once every two weeks.

29. The method of any one of claims 20-28, wherein the BRAF inhibitor is dabrafenib, encorafenib, regorafenib, sorafenib, or vemurafenib.

30. The method of any one of claims 20-29, wherein the BRAF inhibitor is encorafenib.

31. The method of claim 30, wherein encorafenib is administered in an amount that is between about 100 mg/day and about 500 mg/day.

32. The method of claim 30, wherein encorafenib is administered in an amount that is about 450 mg/day.

33. The method of claim 30, wherein encorafenib is administered in an amount that is about 300 mg/day.

34. The method of claim 30, wherein encorafenib is administered in an amount that is about 225 mg/day.

35. The method of claim 30, wherein encorafenib is administered in an amount that is about 150 mg/day.

36. The method of any one of claims 20-29, wherein the BRAF inhibitor is dabrafenib.

37. The method of claim 36, wherein dabrafenib is administered in an amount that is about 150 mg/day.

38. A method of treating cancer in a subject in need thereof, the method comprising: administering to the subject in need thereof a therapeutically effective amount of
(i) compound 1: 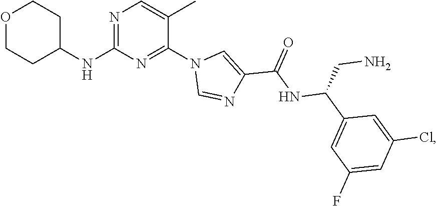 [SMILES: Cc1cnc(NC2CCOCC2)nc1-n1cnc(C(=O)N[C@H](CN)c2cc(F)cc(Cl)c2)c1]
or a pharmaceutically acceptable salt thereof,
(ii) cetuximab; and
(iii) encorafenib.

39. A method of treating cancer in a subject in need thereof, the method comprising administering to the subject in need thereof a therapeutically effective amount of
(i) compound 1: 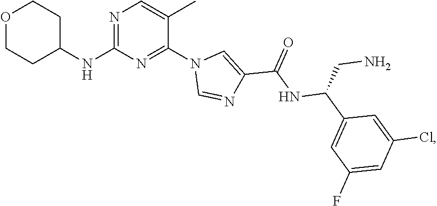 [SMILES: Cc1cnc(NC2CCOCC2)nc1-n1cnc(C(=O)N[C@H](CN)c2cc(F)cc(Cl)c2)c1]
or a pharmaceutically acceptable salt thereof,
(ii) cetuximab; and
(iii) dabrafenib.

40. A method of treating cancer in a subject in need thereof, the method comprising administering to the subject in need thereof a therapeutically effective amount of
(i) compound 1: 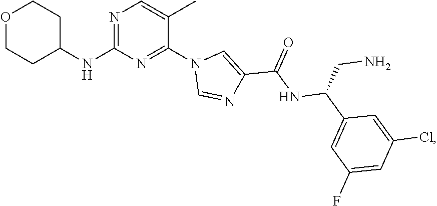 [SMILES: Cc1cnc(NC2CCOCC2)nc1-n1cnc(C(=O)N[C@H](CN)c2cc(F)cc(Cl)c2)c1]
or a pharmaceutically acceptable salt thereof,
(ii) osimertinib; and
(iii) encorafenib.

41. A method of treating cancer in a subject in need thereof, the method comprising administering to the subject in need thereof a therapeutically effective amount of
(i) compound 1: 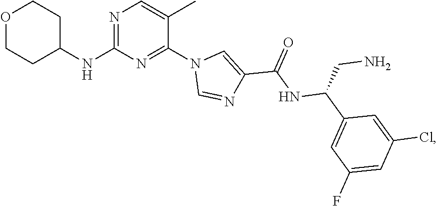 [SMILES: Cc1cnc(NC2CCOCC2)nc1-n1cnc(C(=O)N[C@H](CN)c2cc(F)cc(Cl)c2)c1]
or a pharmaceutically acceptable salt thereof,
(ii) osimertinib: and
(iii) dabrafenib.

42. The method of any one of claims 1-41, wherein the pharmaceutically acceptable salt of compound 1 is the mandelic acid salt.

43. The method of any one of claims 1-42, wherein the cancer is a mitogen-activated protein kinase (MAPK) pathway driven cancer.

44. The method of any one of claims 1-42, wherein the cancer is a BRAF-driven cancer, HRAS-driven cancer, or a NRAS-driven cancer.

45. The method of any one of claims 1-42, wherein the cancer comprises at least one cancer cell driven by deregulated ERK.

46. The method of any one of claims 1-42, wherein the cancer has at least one mutation in RAS.

47. The method of any one of claims 1-42, wherein the cancer has at least one mutation in RAF.

48. The method of any one of claims 1-42, wherein the cancer has at least one mutation in MEK.

49. The method of any one of claims 1-42, wherein the cancer has a G12C KRAS mutation.

50. The method of any one of claims 1-42, wherein the cancer has a G12D KRAS mutation.

51. The method of any one of claims 1-42, wherein the cancer has a G12S KRAS mutation.

52. The method of any one of claims 1-42, wherein the cancer has a G12V KRAS mutation.

53. The method of any one of claims 1-42, wherein the cancer has a G13D KRAS mutation.

54. The method of any one of claims 1-42, wherein the cancer has a Q16H KRAS mutation.

55. The method of any one of claims 1-42, wherein the cancer has a Q16K KRAS mutation.

56. The method of any one of claims 1-42, wherein the cancer has a Q61R NRAS mutation.

57. The method of any one of claims 1-42, wherein the cancer is a BRAF V600E or V600K mutant tumor.

58. The method of any one of claims 1-42, wherein the cancer is a MAPKm/MAPKi-naïve pan cancer.

59. The method of any one of claims 1-42, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

60. The method of any one of claims 1-42, wherein the cancer comprises one or more EGFR mutation selected from the group consisting of L858R, exon 19 deletion, and T790M.

61. The method of any one of claims 1-60, wherein the cancer is a solid tumor.

62. The method of any one of claims 1-61, wherein the cancer is non-small cell lung cancer (NSCLC), melanoma, pancreatic cancer, salivary gland tumor, thyroid cancer colorectal cancer (CRC), or esophageal cancer.

63. The method of any one of claims 1-62, wherein the cancer is non-small cell lung cancer (NSCLC).

64. The method of claim 63, wherein the NSCLC is an EGFR mutant NSCLC.

65. The method of claim 63, wherein the NSCLC is a KRAS G12C mutant NSCLC.

66. The method of claim 63, wherein the NSCLC is a KRAS G12D mutant NSCLC.

67. The method of claim 63, wherein the NSCLC is a KRAS G12S mutant NSCLC.

68. The method of claim 63, wherein the NSCLC is a KRAS G12V mutant NSCLC.

69. The method of claim 63, wherein the NSCLC is a KRAS G13D mutant NSCLC.

70. The method of claim 63, wherein the NSCLC is a KRAS Q61H mutant NSCLC.

71. The method of claim 63, wherein the NSCLC is a KRAS Q61K mutant NSCLC.

72. The method of claim 63, wherein the NSCLC is a NRAS Q61R mutant NSCLC.

73. The method of claim 63, wherein the cancer is a MAPKm/MAPKi-naïve NSCLC.

74. The method of claim 63, wherein the cancer is a BRAFi-treated V600 NSCLC.

75. The method of claim 63, wherein the cancer is a KRAS-treated G12C NSCLC.

76. The method of claim 63, wherein the cancer is a KRAS-treated G12D NSCLC.

77. The method of claim 63, wherein the cancer is a KRAS-treated G12S NSCLC.

78. The method of claim 63, wherein the cancer is a KRAS-treated G12V NSCLC.

79. The method of claim 63, wherein the cancer is a KRAS-treated G13D NSCLC.

80. The method of claim 63, wherein the cancer is a KRAS-treated Q61H NSCLC.

81. The method of claim 63, wherein the cancer is a KRAS-treated Q61K NSCLC.

82. The method of claim 63, wherein the cancer is a NRAS-treated Q61R NSCLC.

83. The method of any one of claims 1-82, wherein the cancer is pancreatic cancer.

84. The method of claim 83, wherein the cancer is a MAPKm/MAPKi-naïve pancreatic cancer.

85. The method of any one of claims 1-62, wherein the cancer is melanoma.

86. The method of claim 85, wherein the melanoma is a BRAF V600E or V600K mutant tumor.

87. The method of claim 85, wherein the cancer is a BRAFi-treated V600 melanoma.

88. The method of any one of claims 1-62, wherein the cancer is salivary gland tumor.

89. The method of any one of claims 1-62, wherein the cancer is thyroid cancer.

90. The method of any one of claims 1-62, wherein the cancer is colorectal cancer (CRC).

91. The method of claim 90, wherein the CRC is a BRAF V600E CRC.

92. The method of claim 90, wherein the CRC is a KRAS mutant CRC.

93. The method of claim 90, wherein the CRC is a KRAS G12C mutant CRC.

94. The method of claim 90, wherein the CRC is a KRAS G12D mutant CRC.

95. The method of claim 90, wherein the CRC is a KRAS G12S mutant CRC.

96. The method of claim 90, wherein the CRC is a KRAS G12V mutant CRC.

97. The method of claim 90, wherein the CRC is a KRAS G13D mutant CRC.

98. The method of claim 90, wherein the CRC is a KRAS Q61H mutant CRC.

99. The method of claim 90, wherein the CRC is a KRAS Q61K mutant CRC.

100. The method of claim 90, wherein the CRC is a NRAS mutant CRC.

101. The method of claim 100, wherein the CRC is a NRAS Q61R mutant CRC.

102. The method of any one of claims 1-62, wherein the cancer is esophageal cancer.

103. The method of any one of claims 1-102, wherein compound 1, or a pharmaceutically acceptable salt thereof, is administered in an amount that is between about 25 mg/day and about 300 mg/day.

104. The method of any one of claims 1-103, wherein compound 1, or a pharmaceutically acceptable salt thereof, is administered in an amount that is between 25 mg/day and 150 mg/day.

105. The method of any one of claims 1-104, wherein compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day, or about 250 mg/day.

106. The method of any one of claims 1-105, wherein compound 1, or a pharmaceutically acceptable salt thereof, is administered in an amount that is about 25 mg/day, about 50 mg/day, about 100 mg/day, or about 150 mg/day.

107. The method of any one of claims 1-105, wherein compound 1, or a pharmaceutically acceptable salt thereof, is administered in an amount that is about 250 mg/day.

108. The method of any one of claims 1-107, wherein compound 1, or a pharmaceutically acceptable salt thereof, is administered once a day (QD).

109. The method of any one of claims 1-107, wherein compound 1, or a pharmaceutically acceptable salt thereof, is administered twice a day (BID).

110. The method of any one of claims 1-107, wherein compound 1, or a pharmaceutically acceptable salt thereof, is administered three times a day (TID).

111. The method of any one of claims 1-110, wherein compound 1, or a pharmaceutically acceptable salt thereof, is administered once a week.

112. The method of any one of claims 1-110, wherein compound 1, or a pharmaceutically acceptable salt thereof, is administered twice a week.

113. The method of any one of claims 1-102, wherein compound 1, or a pharmaceutically acceptable salt thereof, is administered in an amount that is between about 25 mg and about 300 mg twice a day, once a week (BID-QW).

114. The method of any one of claims 1-102, wherein compound 1, or a pharmaceutically acceptable salt thereof, is administered in an amount that is between about 25 mg and about 250 mg twice a day, once a week (BID-QW).

115. The method of any one of claims 1-102, wherein compound 1, or a pharmaceutically acceptable salt thereof, is administered in an amount that is between about 25 mg and about 150 mg twice a day, once a week (BID-QW).

116. The method of any one of claims 1-102, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

117. The method of any one of claims 1-102, wherein compound 1, or a pharmaceutically acceptable salt thereof, is administered in an amount that is about 25 mg, 50 mg, about 100 mg, about 125 mg, or about 150 mg twice a day, once a week (BID-QW).

118. The method of any one of claims 1-102, wherein compound 1, or a pharmaceutically acceptable salt thereof, is administered in an amount that is about 125 mg twice a day, once a week (BID-QW).

119. The method of any one of claims 1-118, wherein compound 1, or a pharmaceutically acceptable salt thereof, is administered for at least one 28-day cycle.

120. The method of any one of claims 1-119, wherein compound 1, or a pharmaceutically acceptable salt thereof, is administered on day 1, day 8, day 15, and day 22 of a 28-day cycle.

121. The method of any one of claims 1-119, wherein compound 1, or a pharmaceutically acceptable salt thereof, is administered on day 1, day 8, day 15 of a 28-day cycle.

122. The method of any one of claims 1-120, wherein compound 1, or a pharmaceutically acceptable salt thereof, is administered orally.

123. The method of any one of claims 1-121, wherein the method further comprises administering an additional MAPK pathway inhibitor.

124. The method of claim 123, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFRIR inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

125. The method of claim 123, wherein the additional MAPK pathway inhibitor is a KRAS inhibitor.

126. The method of claim 123, wherein the additional MAPK pathway inhibitor is a BRAF inhibitor.

127. The method of claim 123, wherein the additional MAPK pathway inhibitor is a EGFR inhibitor.

128. The method of claim 123, wherein the additional MAPK pathway inhibitor is a CDK4/6.

129. The method of claim 123, wherein the additional MAPK pathway inhibitor is a FLT3 inhibitor.

130. The method of claim 123, wherein the additional MAPK pathway inhibitor is adagrasib, afatinib, ASTX029, binimetinib, cobimetinib, dacomitinib, erlotinib, gefitinib, gilteritinib, lapatinib, LTT462, LY3214996, necitumumab, neratinib, nimotuzumab, palbociclib, selumetinib, sotorasib, trametinib, ulixertinib, vandetanib, or vemurafenib.

131. The method of claim 123, wherein the additional MAPK pathway inhibitor is adagrasib.

132. The method of claim 123, wherein the additional MAPK pathway inhibitor is gilteritinib.

133. The method of claim 123, wherein the additional MAPK pathway inhibitor is palbociclib.

134. The method of claim 123, wherein the additional MAPK pathway inhibitor is panitumumab.

135. The method of claim 123, wherein the additional MAPK pathway inhibitor is sotorasib.
